(19) United States
(12) Patent Application Publication
(10) Pub. No.: US 2011/0077300 A1
(43) Pub. Date: Mar. 31, 2011

(54) Metabolic Benefits to Butyrate as a Chronic Diet Supplement

(76) Inventors: Jianping Ye, Baton Rouge, LA (US); Zhangguo Gao, Baton Rough, LA (US)

(21) Appl. No.: 12/731,723

(22) Filed: Mar. 25, 2010

Related U.S. Application Data
(60) Provisional application No. 61/163,548, filed on Mar. 26, 2009.

(51) Int. Cl. A61K 31/22; A61K 31/19; A61K 31/222; A61P 3/10; A61P 3/04; A61P 21/00

(52) U.S. Cl. 514/547; 514/557; 514/546

(57) ABSTRACT

Sodium butyrate was chronically administrated through diet supplementation at 5% w/w in a high fat diet. Supplementation of butyrate prevented development of insulin resistance and obesity in C57BL/6J mice on a high fat diet, and in mice fed on a regular diet of tributyrin. Fasting blood glucose, insulin, and insulin tolerance were all reserved in the butyrate-treated mice. The body fat content was maintained at 10% without a reduction in food intake. Adaptive thermogenesis and fatty acid oxidation were enhanced. An increase in mitochondria function and biogenesis was observed in the skeletal muscle and brown fat, and Type 1 muscle fiber was enriched in the skeletal muscle. In genetically obese mice, supplementation of butyrate led to an increase in insulin sensitivity and reduction in adiposity. Dietary supplementation of butyrate can prevent and treat diet-induced insulin resistance.

This application claims the benefit of U.S. Provisional Application No. 61/163,548, filed Mar. 26, 2009.

The development of this invention was partially funded by the Government under grant numbers DK68036 and P50AT02776-020002 from the National Institutes of Health. The Government has certain rights in this invention.

This invention pertains to the chronic use of butyrate as a dietary supplement to increase insulin sensitivity, increase energy expenditure, and decrease body weight in non-ruminant mammals.

Recent studies suggest that natural compounds represent a rich source for small thermogenic molecules, which hold potential in the prevention and treatment of obesity and insulin resistance. Several natural products, such as resveratrol (1; 2), bile acid (3), and genipin (4), have been reported to increase thermogenic activities in animal or cellular models.

Butyric acid has four carbons in the molecule (CH₃CH₂CH₂—COOH) and becomes sodium butyrate after receiving sodium. Sodium butyrate (SB) is a dietary component found in foods such as cheese and butter. It is also produced in large amounts from dietary fiber after fermentation in the large intestine, where butyric acid is generated together with other short chain fatty acids (SCFAs) from non-digestible carbohydrates, such as non-starch polysaccharides, resistant starch and miscellaneous low-digestible saccharides (5; 6). In addition, butyrate is produced in high amounts in the rumen of ruminant mammals, e.g., sheep, goats, and cattle. Ruminant mammals do not absorb much if any dietary glucose, but produce most serum glucose from fatty acids produced in the rumen. Intravenous use of butyrate, including in the form of tributyrin, in ruminant mammals has been shown to cause an increase in serum glucose and insulin (39;40;41;42). In non-ruminant mammals, dietary sodium butyrate has been reported to increase daily body mass gain in pigs (43). SB has also been shown to have both positive and negative effects on isolated rat liver and islet cells, including decrease in cell viability (islet cells), decrease in insulin release in the presence of glucose (islet cells), and impairment of energy metabolism (liver cells), although there are conflicting reports (44;37). Certain bioactivities of SB has been linked to inhibition of class I and class II histone deacetylases (HDACs) (7). HDACs regulate gene transcription through modification of chromatin structure by deacetylation of proteins including histone proteins and transcription factors.

Dietary intervention is a potential strategy in the prevention and treatment of metabolic syndrome. PGC-1α (peroxisome proliferator-activated receptor γ coactivator 1 alpha), a transcription coactivator, is a promising molecular target in the dietary intervention (1; 2). PGC-1α controls energy metabolism by interaction with several transcription factors, e.g. ERRα, NRF-1, NRF-2, PPARα, PPARδ and thyroid hormone receptor (TR) that direct gene transcription for mitochondrial biogenesis and respiration (8). In the muscle, PGC-1α increases oxidative (Type I) fiber differentiation and enhances fatty acid metabolism (9). In brown fat, PGC-1α stimulates adaptive thermogenesis through up-regulation of UCP-1 expression (10). A reduction in PGC-1α function is associated with mitochondrial dysfunction, reduction in fatty acid oxidation and risk for insulin resistance or type 2 diabetes (11-14). Dietary intervention of PGC-1α activity holds promise in the prevention and treatment of metabolic syndrome. However, knowledge is limited in the dietary components or derivatives that are able to regulate the PGC-1 activity.

We have discovered that chronic administration of dietary butyrate in non-ruminant mammals on a high fat diet will result in lower body weight and prevent diet-induced insulin resistance. In dietary obese C57BL/6J mice, sodium butyrate was administrated through diet supplementation at 5% w/w in the high fat diet (HFD). On the HFD diet, supplementation of butyrate prevented development of insulin resistance and obesity in C57BL/6J mice. Insulin sensitivity was reserved in the butyrate-treated mice as indicated by fasting blood glucose, insulin, insulin tolerance and the clamp test. The body fat content was maintained at 10% without a reduction in food intake. Adaptive thermogenesis and fatty acid oxidation were enhanced, and an increase in mitochondria function and biogenesis was observed in the skeletal muscle and brown fat. The type 1 muscle fiber was enriched in the skeletal muscle. PGC-1α, AMPK and p38 signals were elevated. In genetically obese mice, chronic butyrate supplementation of a regular diet led to an increase in insulin sensitivity and reduction in adiposity. Dietary supplementation of butyrate can prevent and treat diet-induced insulin resistance in mouse.

### BRIEF DESCRIPTION OF DRAWINGS

FIGS. 1A-1I illustrate various metabolic responses to dietary butyrate (5% wt/wt food) in C57BL/6J mice on a high fat diet as compared to control mice. FIG. 1A illustrates the food intake over 10 weeks, each point represents the average daily intake for the previous five days which was converted into K calorie and normalized with body weight (kg) and 24 hours. FIG. 1B illustrates the energy expenditure, measured as K calorie per kilogram lean mass every hour. FIG. 1C illustrates oxygen consumption, measured as ml oxygen in kilogram lean mass per hour. FIG. 1D illustrates the respiratory exchange ratio (RER), a measure of substrate utilization, expressed as a volume ratio of oxygen consumed versus CO₂ exhaled. FIG. 1E illustrates the body weight gain over the 16 weeks. FIG. 1F illustrates the body fat content as a percentage of body weight, as determined by NMR. FIG. 1G illustrates the body muscle content as a percentage of body weight, as determined by NMR. FIG. 1H illustrates the lipid content in feces collected during a 24 hours period at 12 weeks on the high fat diet. (P>0.05, n=5). FIG. 1I illustrates the spontaneous physical activity, measured as the frequency of horizontal movement for day and night time at 10 wks on the high fat diet. For FIGS. 1A-1D and 1I, n=8 in control or butyrate groups; and for 1E-1G, n=10 in control or butyrate groups. Values given are the mean±SE. *P<0.05, **P<0.001 by Student's t test.

FIGS. 2A-2F illustrate the insulin sensitivity in C57BL/6J mice either on a high fat diet (control) or a butyrate-supplemented high fat diet (5% wt/wt food; Butyrate). FIG. 2A illustrates the level of serum glucose collected from tail vein blood after 16 hr fasting over the 16 weeks of feeding. FIG. 2B illustrates the serum insulin level, determined at 16 weeks in a fasting condition with Lincoplex kit (#MADPK-71k-03, Linco Research Inc., St. Charles, Mo.). FIG. 2C illustrates the results of an insulin tolerance test (ITT) in butyrate-treated mice, tested at 12 weeks on HFD (at 16 weeks of age). In FIGS. 2A-2C, data are presented as means±SE (N=9). *P<0.05, **P<0.001 by Student's t test. FIG. 2D illustrates the results of a homeostasis model assessment (HOMA), determined after an overnight fast, and measuring blood glucose and insulin to determine insulin sensitivity through HOMA IR (IR=fasting insulin mU/ml×fasting glucose mg/dL±405). Values are the means±SE (N=8 mice). ** P<0.001. FIG. 2E illustrates the amount of insulin signal in a gastrocnemius muscle isolated 30 min after insulin (0.75 U/kg) injection and used to prepare a whole cell lysate for the immunoblot. Mice on the high fat diet for 13 weeks were used in the signaling assay. FIG. 2F illustrates the quantification of the signal of FIG. 2E, normalization with protein loading. **P<0.001 (n=2).

FIGS. 3A-3H illustrate the response in white adipose tissue in C57BL/6J mice either on a high fat diet (Control) or a butyrate-supplemented high fat diet (5% wt/wt food; Butyrate); and in differentiating adipocytes (3T3-L1 cells). FIG. 3A illustrates the epidermal fat pad in C57BL/6J mice, collected at 13 weeks on HFD (18 weeks in age), and a slide from the fat pad after H&E staining, photographed under a microscope at magnification 20×. FIG. 3B illustrates the gene expression of several proteins in 3T3-L1 cells after induction of cell differentiation, as determined by qRT-PCR at day 8. Butyrate was used at 100 uM during induction of adipogenesis. FIG. 3C illustrates the total triglyceride in 3T3-L1 cells, using Oil red O staining to stain triglyceride in 3T3-L1 mature adipocytes at the day 8 post-induction of differentiation. Butyrate was used at 100 uM during induction of adipogenesis. FIGS. 3D-3H are levels of expression for leptin (FIG. 3D), Adiponectin (FIG. 3E), the macrophage marker F4/80 (FIG. 3F), TNF-α (FIG. 3G), and iNOS (FIG. 3H), determined in the epididymal fat in the C57BL/6J mice. In FIGS. 3A-3H, data are presented as mean±SE (n=8). *P<0.05, **P<0.001, Student t test.

FIGS. 4A-4D illustrate the response in brown adipose tissue response in C57BL/6J mice either on a high fat diet (Control) or a butyrate-supplemented high fat diet (5% wt/wt food; Butyrate). FIG. 4A illustrates the ability to maintain body temperature in mice that were exposed to 4° C. ambient temperature at 10 weeks on the diet regime, measured as rectal temperature. FIG. 4B illustrates brown adipose tissues collected at 13 weeks on the diet regime, used to make tissue slides stained with Hematoxylin and Eosin staining (H&E staining), and photographed at magnification 100×. FIG. 4C illustrates the gene expression for PGC-1α and UCP-1 in brown adipose tissue collected at 13 weeks on the diet regime, measured by qRT-PCR. FIG. 4D illustrates an immunoblot of brown adipose tissues collected at 13 weeks on the diet regime, and used to make a whole cell lysate (100 μg) which was resolved in SDS-PAGE and blotted with PGC-1α and UCP-1 antibodies. In FIGS. 4A, 4C, and 4C, data are presented as means±SE (n=9 mice). * P<0.05.

FIGS. 5A and 5B illustrate proteins in skeletal vastus laterais muscle in C57BL/6J mice either on a high fat diet (Control) or a butyrate-supplemented high fat diet (5% wt/wt food) for 13 weeks. A whole cell lysate was prepared from muscle tissues of each group and analyzed in an immunoblot. Signals for PGC-1α, type I myosin heavy chain (Myosin), myoglobin, phospho-AMPK and phospho-p38 were blotted with specific antibodies. A representative blot is shown in FIG. 5A. The relative signal strength was quantified for each band and shown in FIG. 5B. The results are the mean ±SE (n=8 mice). *P<0.01, ** P<0.001 (vs. control).

FIGS. 6A and 6B illustrate the effect in L6 muscle cells and liver tissue by measuring expression of pAMPK, p-p38 and PGC-1α on an immunoblot using specific antibodies. FIG. 6A illustrates the levels of AMPK and PGC-1α in L6 myotube cells, starved in 0.25% BSA DMEM overnight, and then treated with 500 μM of sodium butyrate for 4 hours. A cell lysate was prepared and analyzed in an immunoblot (FIG. 6A, upper). The relative signal strength was quantified for each band and shown in FIG. 6A, lower, as a mean value for triplicate experiments. FIG. 6B illustrates the levels of AMPK and PGC-1α in liver tissues collected from C57BL/6J mice either on a high fat diet (Control) or a butyrate-supplemented high fat diet (5% wt/wt food) for 13 weeks. The liver was used to make a cell lysate which was analyzed in an immunoblot (FIG. 6B, upper). A representative blot is shown. The relative signal strength was quantified for each band and shown in FIG. 6B, lower, as a mean value five mice (N=5). * P<0.05, **P<0.001.

FIGS. 7A-7G illustrate the increased mitochondrial function in muscles and in L6 muscle cell line. Vastus laterais muscle and blood samples were collected from C57BL/6J mice either on a high fat diet (Control) or a butyrate-supplemented high fat diet (5% wt/wt food) for 13 weeks (18 weeks in age), and examined for fatty acid oxidation, gene expression and blood lipids. FIG. 7A illustrates the fatty acid oxidation in muscle tissue, expressed as a fold change in ¹⁴C-labeled CO₂. FIG. 7B illustrates the gene expression in muscle for PGC-1α, PPARδ, and CPT1b, expressed as a relative fold change in mRNA over the control. FIG. 7C illustrates the relative change in mitochondrial COX I DNA, determined by Sybr green RT-PCR. FIG. 7D illustrates fatty acid oxidation in fully differentiated L6 cells in controls and cells treated with 500 μM butyrate for 16 hours. FIG. 7E illustrates the gene expression in L6 cells for PGC-1α, PPARδ, and CPT1b, expressed as a relative fold change in mRNA over the control. FIG. 7F illustrates the level of butyrate in serum of C57BL/6J mice either on a high fat diet (Control) or a butyrate-supplemented high fat diet (5% wt/wt food) for 13 weeks. FIG. 7G illustrates the level of histone deacetylase activity in muscle isolated from C57BL/6J mice either on a high fat diet (Control) or a butyrate-supplemented high fat diet (5% wt/wt food) for 13 weeks. Data are presented as means±SE (n=6). * P<0.05; ** P<0.001.

FIG. 8 illustrates the level of HDAC inhibition by butyrate and several isoforms of butyrate (butyrate, butyl butyrate, amyl butyrate, isobutyl butyrate, benzyl butyrate, a-methylbenzyl butyrate, hexyl butyrate, heptyl butyrate, pennethyl butyrate, methyl butyrate, and 2-hdroxy-3-methylbutanoic acid), expressed as a percent activity of control HDAC.

FIGS. 9A-9D illustrate metabolic and body changes in C57BL/6J mice fed on HFD for 16 weeks (21 weeks in age) to induce obesity, and then the obese mice were treated with butyrate through food supplement for 5 weeks (5% wt/wt food). FIG. 9A illustrates the body weight in the control and butyrate-treated mice at 0 and 5 weeks of butyrate treatment. FIG. 9B illustrates the fat content as determined by NMR at the end of the 5-week treatment with butyrate. FIG. 9C illustrates the results of an insulin tolerance test (ITT) in mice at the end of the 5-week butyrate experiment, performed after a 4-hour fast. FIG. 9D illustrates the results of homeostasis model assessment (HOMA) determined after an overnight fast, and measuring blood glucose and insulin to determine insulin sensitivity through HOMA IR (IR=fasting insulin mU/ml×fasting glucose mg/dL÷405). Values are the means±SE (N=8 in each group). * P<0.05.

FIGS. 10A-10E illustrate the levels of cholesterol (FIG. 10A), low density lipoprotein (FIG. 10B), high density lipoprotein (FIG. 10C), total triglyceride (FIG. 10D), and inflammation cytokines (TNF-a; FIG. 10E) in the blood from C57BL/6J mice either on a high fat diet (Control) or a butyrate-supplemented high fat diet (5% wt/wt food; Butyrate) for 10 weeks. Data are presented as means±SEM (n=6). **P<0.001 by Student's t test.

FIGS. 11A-11E illustrate various responses in C57BL/6J mice either on a high fat diet (control) or a TSA-supplemented high fat diet (0.6 ug/kg/day; TSA) for 12-13 weeks. FIG. 11A illustrates the body weight of the mice at the end of 12 weeks on the high fat diets. FIG. 11B illustrates an insulin tolerance test conducted in mice (17 weeks of age) at 12 weeks on the high fat diets, and after a 4-hour fast. FIG. 11C illustrates the effect of insulin signaling in a gastrocnemius muscle isolated 30 min after insulin (0.75 U/kg) injection and used to prepare a whole cell lysate for immunoblot. IRS-1 and Akt were examined for tyrosine (Y632) and serine (S473) phosphorylation. FIG. 11D illustrates the result of an immunoblot using whole cell lysate prepared from and assaying for PGC-1α, type I myosin heavy chain (Myosin) and myoglobin with specific antibodies. FIG. 11E illustrates the effect of the cell lysates isolated from gastrocnemius muscle on HDAC activity expressed by the fold change over the control. The results in FIGS. 11A, 11B, and 11E are the mean±SE (n=8 in each group). *P<0.05 (vs. control).

FIGS. 12A-12D illustrate various metabolic and body responses in C57BL/6J mice either on a high fat diet (control) or a butyrate-supplemented high fat diet (2.5% wt/wt food; Butyrate). FIG. 12A illustrates the body weight of mice (16 weeks of age) at 12 weeks on the high fat diets. FIG. 12B illustrates the fat content of the mice (16 weeks of age) at 12 weeks on the high fat diets, as measured by NMR. FIG. 12C illustrates the results of an insulin tolerance test (ITT) in mice (16 weeks of age) at 12 weeks on the high fat diets. FIG. 12D illustrates the results of an glucose tolerance test (GTT) in mice (17 weeks of age) at 13 weeks on the high fat diets, and measured after an overnight fast and after injecting 2.5 g/kg glucose intraperitoneally. Blood glucose was measured at times as indicated. Values are the means±SE (N=8 in each group). * P<0.05.

FIGS. 13A-13E illustrate the results of hyperinsulinemic-euglycemic clamp tests conducted in C57BL/6J mice after being fed for 4 weeks either a high fat diet (control) or a butyrate-supplemented high fat diet (5% wt/wt food; Butyrate). FIG. 13A illustrates the glucose infusion rate (GIR), measured during the last 40 min of clamps. FIG. 13B illustrates the insulin-stimulated glucose uptake in skeletal muscle (gastrocnemius) during the clamp test. FIG. 13C illustrates the insulin-stimulated glucose uptake in white adipose tissue (WAT) during the clamp test. FIG. 13D illustrates the insulin-stimulated glucose uptake in brown adipose tissue (BAT) during the clamp test. FIG. 13E illustrates the hepatic glucose production (HGP) during the clamp test. A radiolabeled tracer was used to determine glucose uptake in the muscle, WAT and BAT. In FIGS. 13A-13E, data are presented as means±SE (N=9). *P<0.05, **P<0.001 by Student's t test.

FIGS. 14A-14E illustrate the metabolic effects of amyl butyrate in C57BL/6J mice either fed a regular chow diet or a chow diet incorporated with amyl butyrate (5 g/kg BW/day) for 4 months. FIG. 14A illustrates the change in body weight for the 4 months. FIG. 14B illustrates the body fat content, determined with NMR. FIG. 14C illustrates the body muscle content, determined as NMR. FIG. 14D illustrates the serum glucose level after overnight fasting. FIG. 14E illustrates an insulin tolerance test (ITT) conducted after 4 hours fasting in mice on the diets for 4 months. Values in FIGS. 14A-14E are the mean±SE (n=7). *P<0.05, **P<0.001 compared to the control by Student's t test.

FIGS. 15A and 15B illustrate the body weight and insulin tolerance in genetically obese mice (ob/ob) when fed either chow diet (control) or chow diet supplemented with tributyrin as a butyrate source. FIG. 15A illustrates the change in body weight after 2 weeks on the experimental diets. FIG. 15B illustrates the results of an insulin tolerance test (ITT) conducted after 4 hours fasting in mice on the diets for 6 weeks. Values in FIGS. 15A-15B are the mean±SE (n=8). *P<0.05, compared to the control by Student's t test.

Sodium butyrate, a salt of butyric acid (a short chain fatty acid), was examined in the regulation of insulin sensitivity in mice fed a high fat diet over a span of sixteen weeks. In response to SB, food intake in the mice was increased in the absence of a gain in body weight. Energy expenditure was enhanced in these mice. Adaptive thermogenesis and fatty acid oxidation were enhanced by butyrate without an increase in spontaneous physical activity. Hepatic steatosis and adipose chronic inflammation were both reduced. Insulin sensitivity was not reduced by the high fat diet with SB. Mitochondrial function and biogenesis were enhanced in brown adipose tissue and skeletal muscle. The mice also had an increase in Type I muscle fibers in skeletal muscle. PGC-1α expression was elevated at mRNA and protein levels together with mitochondrial genes (UCP-1, CPT-1b and COX-I). Taken collectively, the data indicate that butyrate improves insulin sensitivity through energy expenditure and that sodium butyrate is a regulator of PGC-1α. Butyrate is a known inhibitor of histone deacetylase (HDAC). Another HDAC inhibitor, trichostatin A (TSA) was also tested, and caused similar effects in the mice as butyrate. Without wishing to be bound by this theory, we believe that the increase in PGC-1α is probably due to the inhibition of HDACs which control many transcription factors. This is the first report of an inhibitor of HDAC having an effect on PGC-la activity in mice. A triglyceride of butyrate (tributyrin) was also tested in ob/ob mice with results similar to the sodium butyrate salt. The weight gain in the ob/ob mice was significantly reduced and insulin sensitivity was preserved by tributyrin.

### EXAMPLE 1

Materials and Methods

Animal model. Male C57BL/6J (4 weeks in age) and ob/ob mice (4 weeks in age) were purchased from the Jackson Laboratory (Bar Harbor, Me.). After one week quarantine, the C57BL/6J mice were fed on the high fat diet (HFD, D12331, Research Diets, New Brunswick, N.J.), which contains 58% calories in fat. The ob/ob mice were fed on Chow diet (5001, Lab Diet) that contains 13.4% calories in fat. All of the mice were housed in the animal facility with a 12:12-h light-dark cycle and constant temperature (22-24° C.). The mice were free to access water and diet. All procedures were performed in accordance with National Institute of Health guidelines for the care and use of animals and approved by the Institute Animal Care and Use Committee at the Pennington Biomedical Research Center.

Sodium butyrate (SB) administration. Sodium butyrate was administrated through dietary supplementation. Sodium butyrate (#303410, Sigma) was incorporated into the HFD evenly through blending the diet and sodium butyrate together at 400 rpm in a food processor. Sodium butyrate (50 g) was used in 1 kg diet to reach a dose of 5% w/w. The sodium butyrate containing diet was pelleted and stored in a −20° C. freezer. Trichostatin A (TSA) (T-1052, A.G. Scientific Inc. San Diego, Calif.) was dissolved in DMSO, and diluted in 0.9% saline at 2:98 ratio. The TSA solution (200 ul) was administrated by intra-peritoneal (i.p.) injection to reach a dose of 0.6 g/kg/day. The control mice were administrated with the same volume of vehicle. 50 ml of tributyrin (#113026, Sigma) was incorporated into one kilogram of Chow diet in powder to reach a dose of 5% w/w in mice.

Intraperitoneal insulin tolerance (ITT). ITT was conducted by intra-peritoneal (i.p.) injection of insulin (19278, Sigma) at 0.75 U/kg body weight in mice after a 4 hour fast as previously described (15). Blood glucose was monitored in the tail vein blood using the FreeStyle blood glucose monitoring system (TheraSense, Phoenix, Ariz.).

Nuclear magnetic resonance. Body composition was measured using quantitative nuclear magnetic resonance (NMR) as previously described (15). In the test, a conscious and unrestrained mouse was placed in a small tube one at a time and individually tested using a Brucker model mq10 NMR analyzer (Brucker, Canada, Milton ON, Canada). The fat and lean mass were recorded within 1 min. Measurements were made in triplicate for each mouse.

Hyperinsulinemic-euglycemic clamp. Hyperinsulinemic-euglycemic clamps were performed at the Penn State Mouse Metabolic Phenotyping Center. The clamps were conducted in C57BL/6 mice at 12 weeks of age after 4 weeks on HFD with or without butyrate supplement. Following overnight fast (˜15 hour), a 2-hour hyperinsulinemic-euglycemic clamp was conducted in awake mice with a primed (150 mU/kg body weight) and continuous infusion of human regular insulin (Humulin; Eli Lilly, Indianapolis, Ind.) at a rate of 2.5 mU/kg/min to raise plasma insulin within a physiological range (Kim et al., 2004). Blood samples (20 μl) were collected at 20 min intervals for the immediate measurement of plasma glucose concentration, and 20% glucose was infused at variable rates to maintain glucose at basal concentrations. Basal and insulin-stimulated whole body glucose turnover were estimated with a continuous infusion of [3-³H]glucose (PerkinElmer, Boston, Mass.) for 2 hours prior to the clamps (0.05 μCi/min) and throughout the clamps (0.1 μCi/min), respectively. To estimate insulin-stimulated glucose uptake in individual tissues, 2-deoxy-D-[1-¹⁴C]glucose (2-[¹⁴C]DG) was administered as a bolus (10 μCi) at 75 min after the start of clamps. Blood samples were taken before, during, and at the end of clamps for the measurement of plasma [³H]glucose, ³H₂O, 2-[¹⁴C]DG concentrations, and/or insulin concentrations. At the end of the clamps, mice were euthanized, and tissues were taken for biochemical and molecular analysis.

Glucose concentrations during clamps were analyzed using 10 μl plasma by a glucose oxidase method on a Beckman Glucose Analyzer 2 (Beckman, Fullerton, Calif.). Plasma insulin concentrations were measured by ELISA using kits from Alpco Diagnostics (Salem, N.H.). Plasma concentrations of [3-³H]glucose, 2-[¹⁴C]DG, and ³H₂O were determined following deproteinization of plasma samples as previously described. The radioactivity of ³H in tissue glycogen was determined by digesting tissue samples in KOH and precipitating glycogen with ethanol. For the determination of tissue 2-[¹⁴C]DG-6-P content, tissue samples were homogenized, and the supernatants were subjected to an ion-exchange column to separate 2-[¹⁴C]DG-6-P from 2-[¹⁴C]DG. Rates of basal hepatic glucose production (HGP) and insulin-stimulated whole body glucose turnover were determined as the ratio of the [³H]glucose infusion rate to the specific activity of plasma glucose at the end of the basal period and during the final 30 min of clamp, respectively (Kim et al., 2004). Insulin-stimulated rate of HGP during clamp was determined by subtracting the glucose infusion rate from whole body glucose turnover. Insulin-stimulated glucose uptake in individual tissues was assessed by determining the tissue content of 2-[¹⁴C]DG-6-P and plasma 2-[¹⁴C]DG profile.

Quantitative real-time RT-PCR. Total RNA was extracted from frozen tissues (kept at −80° C.) using Tri-Reagent (T9424, Sigma) as described elsewhere (16). Taqman RT-PCR primer and probe were used to determine mRNA for PGC-1α (Mm00447183_m1), UCP-1 (Mm00494069_m1), PPARδ (Mm01305434_m1) and CPT1b (Mm00487200_m1). The reagents were purchased from Applied Biosystems (Foster City, Calif.). Mouse ribosome 18S rRNA_s1 (without intron-exon junction) was used as an internal control. Reaction was conducted with 7900 HT Fast real time PCR System (Applied Biosystems, Foster City, Calif.).

Metabolic chamber. Energy expenditure, respiratory exchange ratio (RER), spontaneous physical movement, and food intake were measured simultaneously for each mouse with the Comprehensive Laboratory Animal Monitoring System (Columbus Instruments, Columbus, Ohio) as described previously (15). The temperature in the metabolic chamber was 24° C. The mice were housed individually in the metabolic chamber. After 48 h of adaptation, the data were recorded for all parameters and used in analysis of the energy metabolism.

Body temperature in cold response. Body temperature was measured in the cold room with ambient temperature at 4° C. Animals were sedated and restrained for less than 30 sec in the measurement. A Thermalert model TH-8 temperature monitor (Physitemp, Clifton, N.J.) was used with probe placed in the rectum at 2.5 cm in depth.

Western Blotting. Fresh fat and muscles were collected and frozen in liquid nitrogen. The whole cell lysate protein was extract in lysis buffer with sonication and analyzed in western blot as described elsewhere (16). Antibodies were used in study of myoglobin (sc-25607, Santa Cruz), PGC-1 (sc-13068, Santa Cruz), Tubulin (ab7291, Abcam), myosin (M8421, Sigma), pAMPK (Thr 172, #2531, Cell signaling) and p-p38 (sc-7975, Santa Cruz). To detect multiple signals from one membrane, the membrane was stripped with a stripping buffer. Intensity of the immunoblot signal was quantified using a computer program, ImageJ 1.37v (NIH). The mean values of results from three experiments (6 mice each group) were presented. The antibodies to PGC-1α and UCP-1 were from Dr. Thomas Gettys at Pennington Biomedical Research Center, Baton Rouge, La.

Muscle fiber type. The fiber types in skeletal muscle were examined using two methods: succinate dehydrogenase (SDH) staining for ATPase and immunostaining of type I myosin heavy chain. In the SDH staining, mid-belly cross-sections of muscle were cut at 8 μm in a cryostat (−20° C.). After drying for 5 min at room temperature, the sections were incubated at 37° C. for 60 min in the incubation solution containing 6.5 mmol/l sodium phosphate monobasic, 43.5 mmol/l sodium phosphate biphasic, 0.6 mmol/l nitroblue tetrazolium (74032, Sigma), and 50 mmol/l sodium succinate (14160, Sigma). The sections were rinsed three times (30 sec/time) in 0.9% saline, 5 min in 15% ethanol and then mounted with aqueous mounting medium (Dakocyrtoma).

Immunohistostaining. Fresh skeletal muscle was collected, embedded in gum tragacanth mixed with OCT freezing matrix, and quickly frozen in isopentane cooled in liquid nitrogen. The tissue slides were obtained through serial cross-section cutting at 8 um thickness and processed with a standard procedure. The slides was blotted with a monoclonal antibody against the type I myosin heavy chain (M8421, Sigma) at 1:200 dilution. After being washed, the slide was incubated with a biotinylated secondary antibody (BA-2000). For PGC-1 staining, paraffin sections (8 μm) of BAT or inguinal fat on slides were deparaffinized and blotted with primary antibody of PGC-1 (sc-13067, Santa Cruz) at 1:200, the sections were washed and incubated with a biotinylated secondary antibody (rabbit IgG) in ABC kit. The slides were then incubated with the ABC elite reagent (PK-6101) and color reaction was performed using the DAB substrate kit (SK-4100) for myosin I and AEC substrate kit (AEC101, Sigma) for PGC-1a according to instructions by the manufacturers.

Hematoxylin and Eosin (H&E) staining. Fresh tissues (muscle, fat and liver) were collected at 16 weeks of age after 12 weeks butyrate feeding and fixed in 10% neutral buffered formalin solution (HT50-1-2, Sigma). The tissue slides were obtained through serial cross-section cutting at 8 um thickness and processed with a standard procedure. Briefly, the slides were deparaffinated and stained in haematoxylin (#101542, Surgipath) for 15 min, and rinsed in water until sections are blue. Then, slides were stained in Eosin (E4009, Sigma), dehydrated quickly in 95% ethanol and treated with phenazine methosulfate. The sections were mounted and photographed with Nikon microscope (Eclipse TS100, Japan).

Histone deacetylase assay and nuclear extract preparation. Histone deacetylase assay were conducted according to the instruction from the manufacturer (#17-320, Upstate). Briefly, 10 ug of muscle nuclear extract (as an enzyme) was incubated with [3H]-acetyl CoA (#TRK688, Amersham) radio labeled histone H4 peptide (25,000 CPM, as a substrate) at 37° C. for 12 hours by shaking Released [3H]-acetate was measured using a scintillation counter. The nuclear extract was prepared according to a protocol described elsewhere (17). The muscle tissues were collected and snap-frozen in liquid nitrogen within 2 min of cervical dislocation of mice. Tissue samples were stored at −80° C. until further processing. The muscle sample of 200˜300 mg was cut into small pieces on dry ice and homogenized in 1 ml of lysate buffer. After centrifugation at 10,000 rpm for 1 minute at 4° C., the nucleus was pelleted and collected. After being washed, the nucleus pellet was treated with extraction buffer. The supernatant was collected for nuclear protein after centrifugation at 14,000 rpm for 5 minutes at 4° C.

Lipids in serum and feces: The serum fatty acids including butyrate were examined using a protocol described elsewhere (18), and as described below in Example 10. The fatty acids in feces were determined using a protocol as described by Schwarz (19). Triglyceride and cholesterol were measured in the whole blood with the Cardiochek portable test system.

Statistical Analysis. In this study, the data were presented as mean±SE from multiple samples. All of the in vitro experiments were conducted three times at least. Student's t test or two-way ANOVA was used in the statistical analysis with significance P≦0.05.

### EXAMPLE 2

Effect of Butyrate on Energy Metabolism.

Butyrate was initially tested for prevention of dietary obesity. In the diet-induced obesity model, the butyrate supplementation started at the beginning of high fat diet (HFD) feeding. The plain HFD was used in the control group. Calorie intake was monitored four times in the first ten weeks. After normalization with body weight, the calorie intake was reduced with the increase in age.

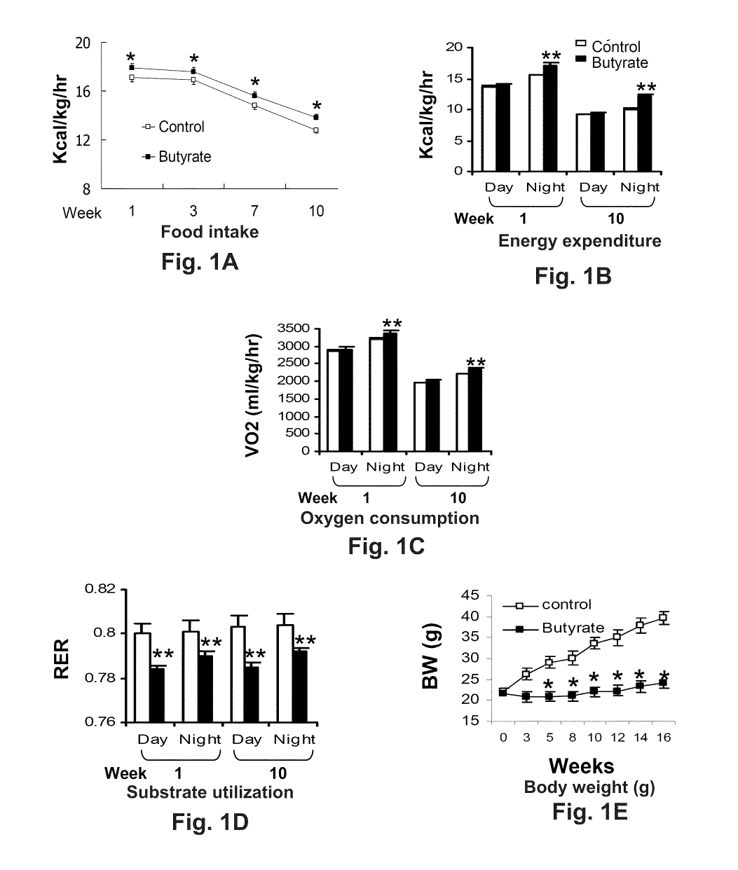

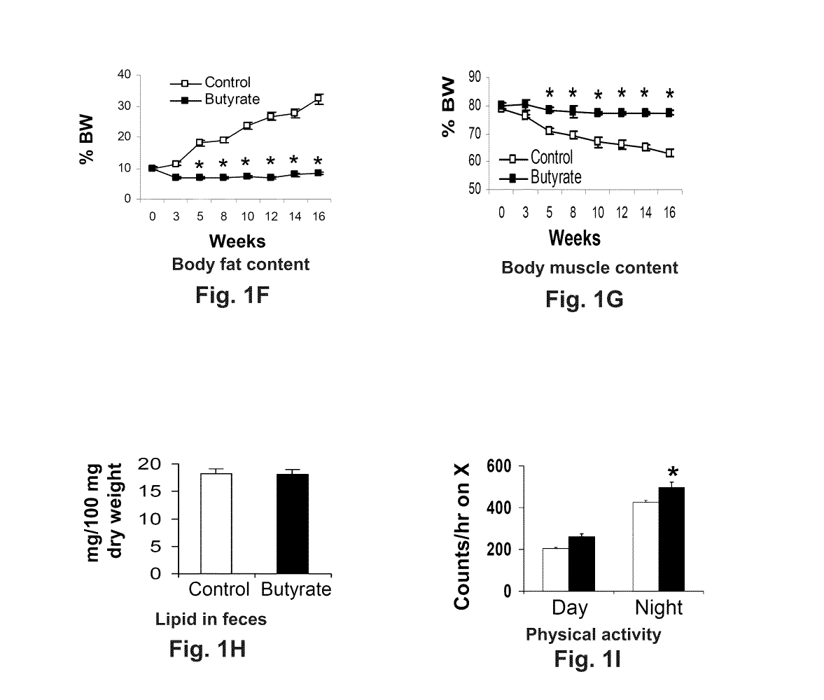

Energy expenditure was examined in C57BL/6J mice using the metabolic chamber at the first week and the tenth week on HFD (16 weeks in age). In this study, sodium butyrate was used at 5% w/w in HFD. FIG. 1A shows the food intake for the 10 weeks. Food intake was monitored daily for 5 days at each time point. Average daily food intake (g) was converted into K calorie and normalized with body weight (kg) and 24 hours. FIG. 1B shows the energy expenditure of the control and butyrate-fed mice at weeks 1 and 10, expressed as K calorie per kilogram lean mass every hour. FIG. 1C shows the oxygen consumption of the control and butyrate-fed mice at weeks 1 and 10, expressed as ml volume oxygen in kilogram lean mass per hour. FIG. 1D shows the substrate utilization of the control and butyrate-fed mice at weeks 1 and 10, expressed by respiratory exchange ratio (RER), which is a volume ratio of oxygen consumed versus CO₂ exhaled. FIG. 1E illustrates the body weight of the mice over 16 weeks. FIG. 1F illustrates the body fat content as determined by NMR expressed as a percentage of body weight. FIG. 1G illustrates the body muscle content expressed as a percentage of body weight. FIG. 1H shows the amount of lipid in feces. Feces were collected in the cages during a 24 hr period on HFD at 12 weeks, and the total lipids were extracted and quantified (P>0.05, n=5). FIG. 1I shows the spontaneous physical activity. The frequency of horizontal movement was shown for day and night time at 10 wks on HFD. For FIGS. 1A-1D and 1I, the number of mice was 8 in both the control or butyrate groups. For FIGS. 1E-1G, the number of mice was 10 in both the control or butyrate group. Values given in FIGS. 1A-1I are the mean±SE. *P<0.05, **P<0.001 by Student's t test.

In the butyrate group, calorie intake was significantly higher at all of the time points (FIG. 1A). The energy expenditure, oxygen consumption and substrate utilization were monitored using the metabolic chamber. In the butyrate group, the energy expenditure and oxygen consumption were elevated at the night time (FIGS. 1B and 1C). The respiratory exchange ratio (RER) was reduced during the day and night time (FIG. 1D), indicating an increase in the fatty acid oxidation in response to butyrate. These data indicate that SB increased energy expenditure in the diet-induced obesity.

Body weight and fat content was monitored in the study. In the control mice, the body weight was increased from 23 g to 40 g after 16 weeks on HFD (FIG. 1E), and the fat content (adiposity) was increased from 10% to 35% of the body weight (FIG. 1F). Accordingly, the lean mass was reduced from 80% to 65% (FIG. 1G). In the butyrate group, these parameters were not significantly changed during the 16 weeks on HFD (FIGS. 1E, 1F, and 1G), indicating that butyrate prevented diet-induced obesity. Mouse growth was not influenced by butyrate as the body length was identical between the two groups. Dietary fat digestion and absorption in the gastrointestinal track was examined by measuring the fatty acid content in feces. The fat content was identical in the feces of two groups (FIG. 1H), indicating that butyrate does not influence fat absorption by the gastrointestinal track. Spontaneous physical activity was monitored in daytime and nighttime in the mice. The data indicate that the physical activity was not reduced by butyrate (FIG. 1I). An increased activity was observed in the butyrate group at the night time. These data indicated that dietary supplementation of butyrate protected the mice from diet-induced obesity. This effect was associated with an increase in energy expenditure and fatty acid oxidation. The food intake and physical activity indicated that no toxicity was observed for butyrate in the mice.

### EXAMPLE 3

Effect of Dietary Butyrate on Insulin Sensitivity.

The increase in energy metabolism shown in Example 2 indicated that butyrate may protect the mice from HFD-induced insulin resistance. To test this possibility, systemic insulin sensitivity was analyzed using fasting glucose, insulin, and insulin tolerance.

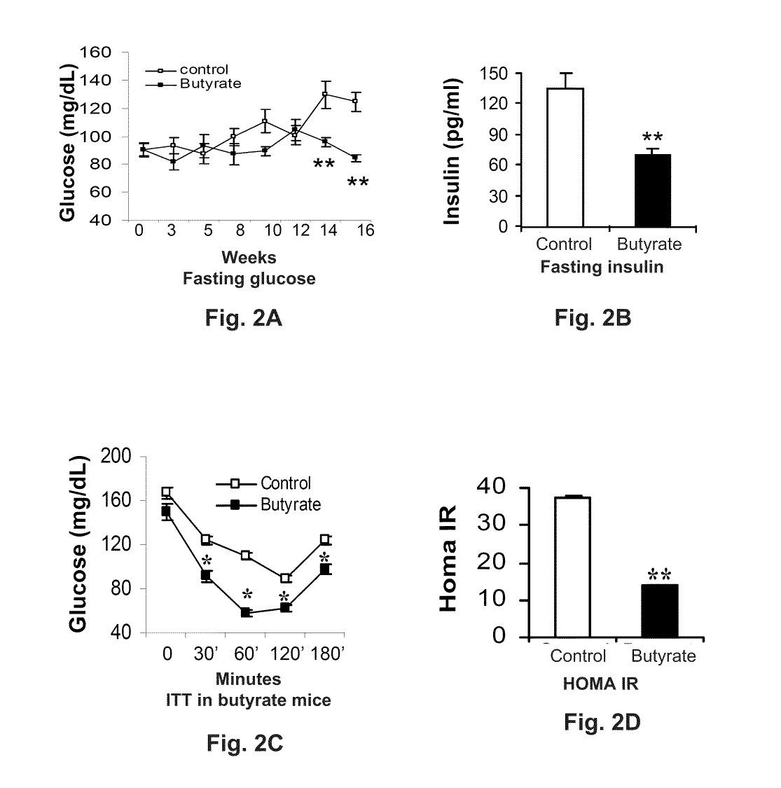

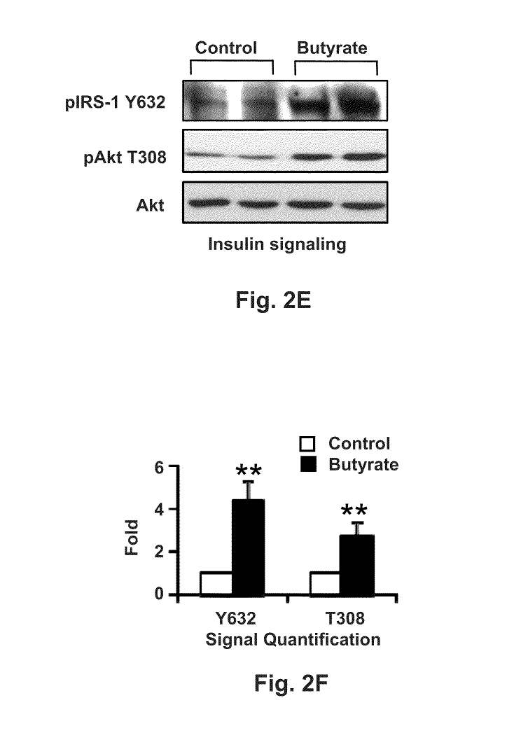

The results for testing insulin sensitivity in butyrate-treated mice are shown in FIGS. 2A-2F. FIG. 2A shows the fasting serum glucose levels. Tail vein blood was used for the glucose assay after 16 hr fasting during the period of HFD feeding. FIG. 2B shows the fasting level of serum insulin. The insulin was determined at 16 weeks on HFD in fasting condition with Lincoplex kit (#MADPK-71k-03, Linco Research Inc., St. Charles, Mo.). FIG. 2C shows intraperitoneal insulin tolerance (ITT) in butyrate-treated mice, conducted by intra-peritoneal (i.p.) injection of insulin (19278, Sigma) at 0.75 U/kg body weight in mice after a 4-hour fast. ITT was done at 12 weeks on HFD (at 16 weeks of age). In FIGS. 2A-2C, data are presented as means±SE (N=9). *P<0.05, **P<0.001 by Student's t test. FIG. 2D shows the homeostasis model assessment (HOMA). After an overnight fast, blood glucose and insulin were measured and used to determine insulin sensitivity through HOMA IR (IR=fasting insulin mU/ml×fasting glucose mg/dL÷405). Values are the means±SE (N=8 mice). ** P<0.001. FIG. 2E shows the level of insulin signaling. The gastrocnemius muscle was isolated after insulin (0.75 U/kg) injection in mice for 30 min and used to prepare the whole cell lysate for an immunoblot. The mice on HFD for 13 weeks were used in the signaling assay. FIG. 2F illustrates the degree of the blot signal in the panel 2E, by quantifying the signal and normalizing with protein loading. **P<0.001 (n=2).

In the control group, the fasting glucose was increased significantly after 10 weeks on HFD (FIG. 2A). In the butyrate group, this increase was not observed (FIG. 2A). The fasting insulin was 50% lower in the butyrate group at 16 weeks on HFD (FIG. 2B). In the ITT test, the butyrate group exhibited much better response to insulin at all time points (30, 60, 120 and 180 min) (FIG. 2C). HOMA-IR was 60% lower in the butyrate group (FIG. 2D). These data indicate that insulin resistance was prevented in the butyrate group. Insulin signaling was examined in the skeletal muscle lysate with tyrosine 632 (Y632) phosphorylation of IRS-1 protein and threonine 308 phosphorylation of Aid (FIG. 2E). Both signals were increased in the butyrate-treated mice (FIGS. 2, E and F), indicating a potential molecular mechanism of insulin sensitization.

### EXAMPLE 4

Effect of Dietary Butyrate on White Adipose Tissue (WAT).

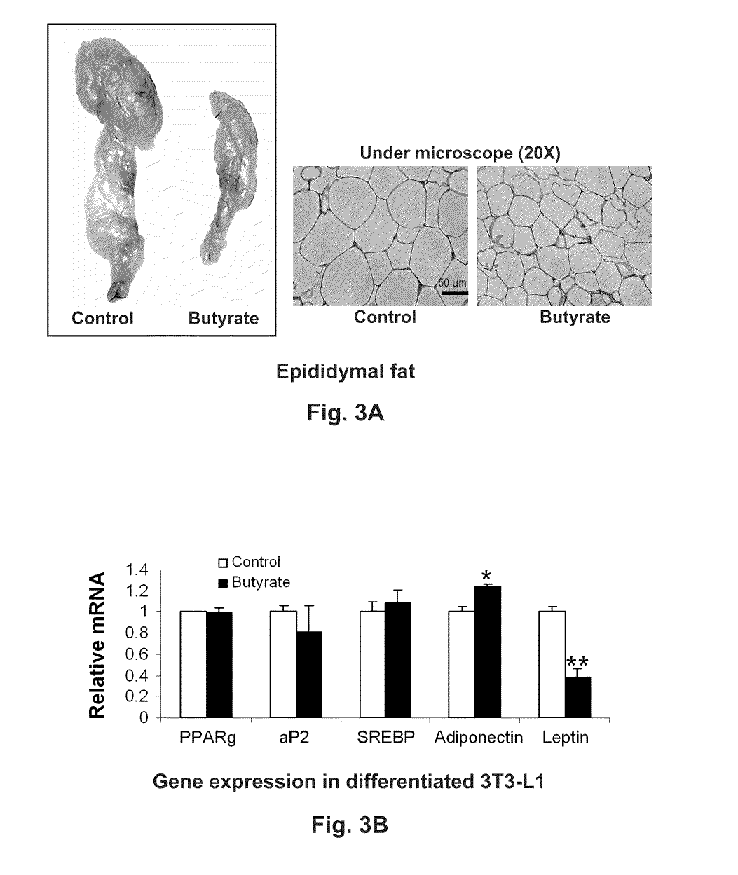

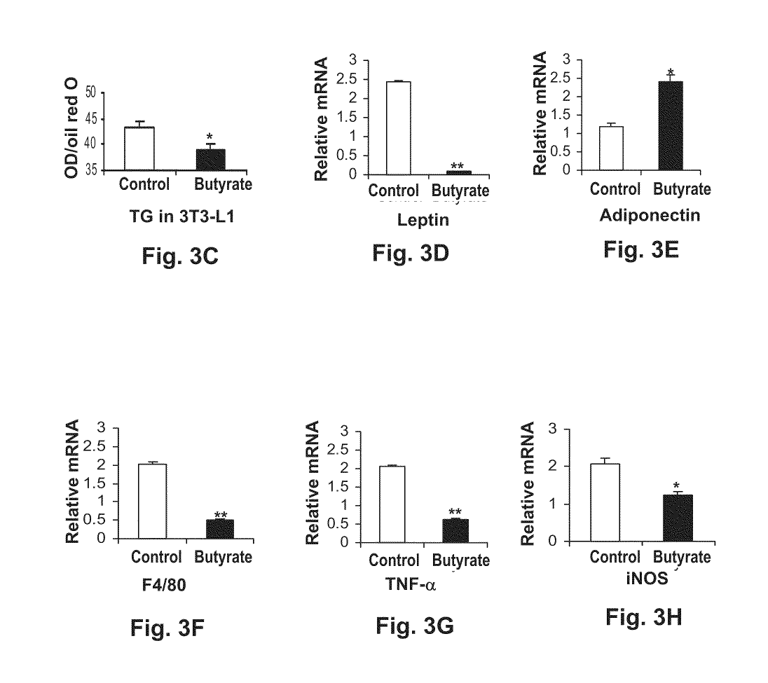

The difference in whole body adiposity in the two groups of mice as shown above indicated that butyrate may reduce obesity by reducing white adipose tissue mass in the body. The epididymal fat pad was examined to test this effect of butyrate. The tissue was collected at 13 weeks on HFD (18 weeks in age) and used to make a tissue slide (FIG. 3A). After H&E staining, the slide was photographed under a microscope at magnification 20× (FIG. 3A). Gene expression was determined by qRT-PCR at day 8 after induction of cell differentiation of 3T3-L1 cells. Butyrate was used at 100 uM during induction of adipogenesis. The results are shown in FIG. 3B. Triglycerides in the 3T3-L1 mature adipocyte cells were quantified using Oil red O staining at the day 8 post-induction of differentiation (FIG. 3C). In addition, levels of mRNA for leptin, adiponectin, F4/80 (macrophage marker), TNF-α, and iNOS were determined in the epididymal fat and their relative levels are presented in bar figures (FIGS. 3D-3H). In FIGS. 3A-3H, data are represented as mean±SE (n=8). *P<0.05, **P<0.001.

In comparison to the control mice on HFD, the mass of fat pad and size of adipocytes were significantly smaller in the butyrate-treated mice (FIG. 3A). The fat pad was 80% less in mass and adipocyte size was 50% smaller than those of the control mice (FIG. 3A). The inguinal fat was also examined and a similar difference was observed between the butyrate-treated mice and control mice (data not shown). The data indicates that butyrate is able to reduce triglyceride accumulation in adipocytes.

To confirm the butyrate effect on adipocytes, 3T3-L1 cells were used to study butyrate activity in vitro. The cells were treated with butyrate during induction of adipogenesis, which was determined by expression of adipocyte-specific genes and triglyceride amount. The results suggest that butyrate does not inhibit adipogenesis as the adipocyte-specific markers (PPARδ, aP2, SREBP, and adiponectin) were not reduced by butyrate (FIG. 3B). However, expression of leptin was reduced by butyrate (FIG. 3B). The reduction may be a result of a decrease in triglyceride accumulation in the differentiated 3T3-L1 adipocytes (FIG. 3C). The triglyceride level was reduced 50% in the cells treated by butyrate.

Endocrine and inflammation activities were investigated in the epididymal fat pads by gene expression. Compared to the control mice on HFD, the butyrate group expressed a lower level of leptin (FIG. 3D), which is consistent with reduced leptin expression in differentiated 3T3-L1 adipocytes after butyrate treatment (FIG. 3B). However, expression of adiponectin was 50% higher (FIG. 3E). Inflammation was assessed by macrophage marker (F4/80), and inflammation cytokines, such as TNF-α and iNOS (inducible nitric oxide synthase) (FIGS. 3F-3H). Expression of these genes was reduced by butyrate. These data indicate that butyrate may prevent chronic inflammation in white adipose tissue in mice fed on HFD. This may be a result of reduced adiposity in the butyrate-treated mice.

### EXAMPLE 5

Effect of Dietary Butyrate on Brown Adipose Tissue (BAT).

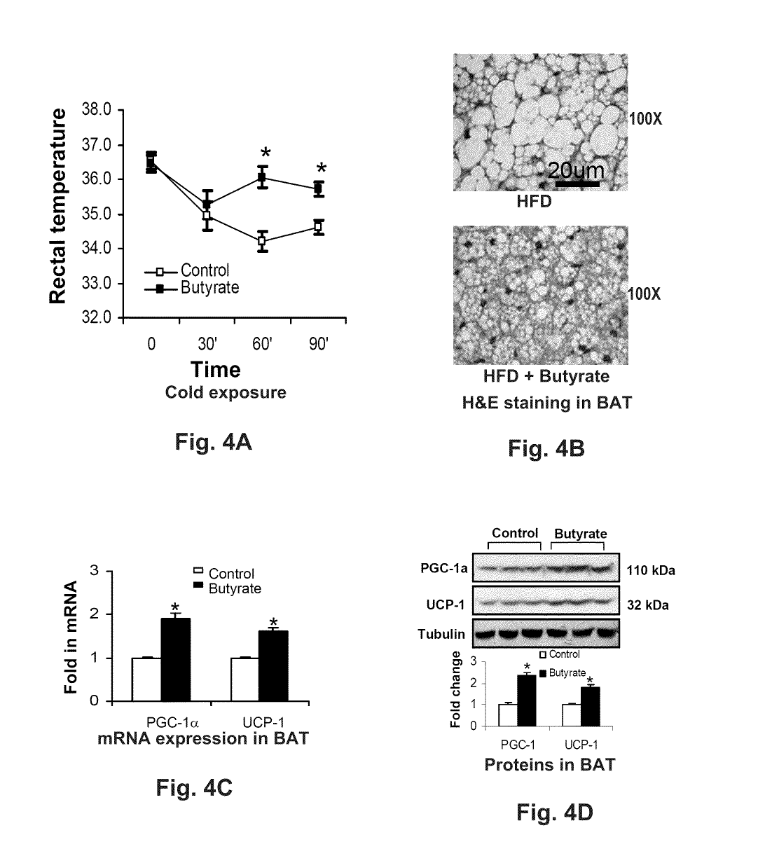

The association of increased food intake with elevated energy expenditure implicated a role of brown adipose tissue, which is responsible for adaptive thermogenesis in response to diet or cold (20-22). Diet-induced thermogenesis reduces obesity in both humans and animals (23). In the butyrate group, the increase in energy expenditure was observed at night when mice actively took food (FIGS. 1A and 1B), which indicated an increase in thermogenesis. To determine the thermogenic function, a cold-response experiment was conducted by exposing the mice to a cold environment with an ambient temperature of 4° C. for 90 min. The mice had been on HFD for 10 weeks. The body core temperature was monitored three times during exposure by measuring the rectal temperature. In the control mice, the body temperature decreased with time and was 34.5° C. after 90 min in the cold (FIG. 4A). In the butyrate-treated mice, the body temperature dropped to 35° C. transiently at 30 minutes, and then returned to 36° C. for the rest of time. These data show that the thermogenic function is enhanced in the butyrate group.

In addition, the morphology and gene expression were examined in the brown fat of mice. Hematoxylin and eosin staining (H&E staining) was conducted on brown adipose tissues collected at 13 weeks on HFD. As shown in FIG. 4B, a photograph was taken at magnification 100×. Compared to the control mice, the size of brown adipocytes was much smaller in the butyrate group (FIG. 4B), indicating a higher thermogenic activity that leads to the reduction in fat accumulation. Mitochondrial function is regulated by gene expression (24). To understand the molecular basis of the increased thermogenesis, the expression of two thermogenesis-related genes, such as PGC-1α and UCP-1, were examined in BAT. mRNA of both genes was increased in the butyrate-treated mice. Brown adipose tissues were collected at 13 weeks on HFD, and gene expression for PGC-1α and UCP-1 was examined by qRT-PCR. The results are shown in FIG. 4C. In addition, the corresponding proteins were assayed using a whole cell lysate (100 μg) resolved in SDS-PAGE and blotted with PGC-1α and UCP-1 antibodies. As shown in FIG. 4D, the corresponding proteins were also increased in the brown fat of the butyrate mice. Data in FIGS. 4A-4D are presented as means±SE (n=9 mice). * P<0.05. This increase in gene expression provides a molecular basis for the enhanced thermogenesis by the butyrate treatment.

### EXAMPLE 6

Effect of Dietary Butyrate on Fiber Type in Skeletal Muscle.

To further understand the cellular basis of enhanced fatty acid utilization in the butyrate group, the skeletal muscle fiber types were assessed. PGC-1α was reported to induce transformation of skeletal muscle fiber from glycolytic type (Type II) into oxidative type (Type I) in transgenic mice (9). Type I fibers are distinct from Type II fibers in several properties (25). Type I fibers (oxidative and slow-twitch fibers) are rich in mitochondria, red in color, and actively use fat oxidation for ATP biosynthesis. Type II fibers (glycolytic and fast-twitch fibers) are relatively poor in mitochondria activity, lighter in color, and depend on glycolysis for ATP production. The butyrate effect on PGC-1α in BAT suggests that skeletal muscle fibers may be changed by butyrate.

To look for oxidative fiber in skeletal muscle, the vastus laterais muscle was isolated from mice that were fed on HFD for 13 weeks. Compared to the control group, the butyrate group exhibited a deep red color (picture not shown). A fiber type analysis was conducted in the vastus laterais, gastrocnemius (rich in glycolytic fibers) and soleus (rich in oxidative fiber). Serial cryostat sections of muscle were made from vastus lateralis, gastrocnemius (gastr.) and soleus muscle. The slides were stained with antibody against Type I myosin heavy chain for oxidative fibers. The ratio of type I fibers were increased in all of the skeletal muscle of butyrate-treated mice (data not shown). Succinate dehydrogenase staining of oxidative fibers was done on serial cryostat sections of the vastus lateralis and gastrocnemius (gastr.) muscle. Again, the butyrate mice showed an increase in Type I fibers (data not shown).

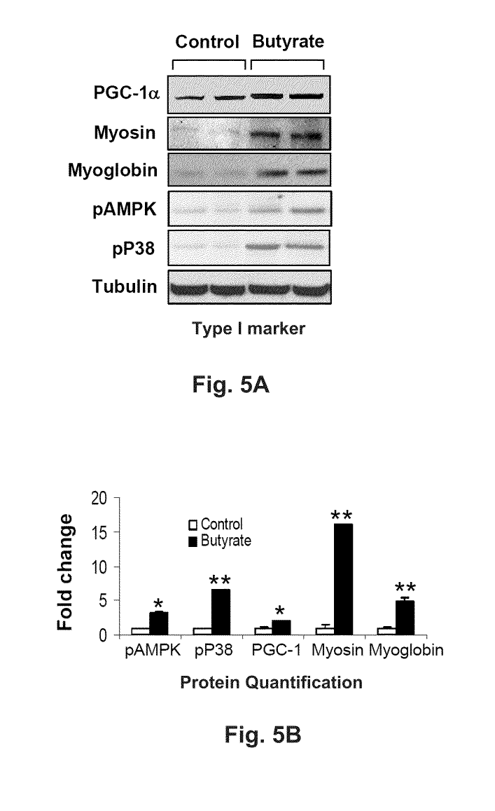

To quantify the amount of protein of type 1 myosin heavy chain and PGC-1α, whole cell lysates were prepared from muscle tissues and analyzed in an immunoblot. Signals for PGC-1α, type I myosin heavy chain (Myosin), myoglobin, phospho-AMPK and phospho-p38 (p-p38) were blotted with specific antibodies. A representative blot is shown in FIG. 5A. Relative signal strength was quantified for each band and expressed in FIG. 5B. The results are the mean±SE (n=8 mice). *P<0.01, ** P<0.001 (vs. control). A significant increase was observed in both proteins in the butyrate-treated mice (FIGS. 5A and 5B). Myoglobin (another marker of oxidative type 1 fiber) was also increased by butyrate (FIG. 5A). A mean value of each protein was presented in the bar figure in FIG. 5B. These data suggest that the ratio of type I fiber was increased by butyrate in the skeletal muscle.

AMPK and p38 activities were examined by their phosphorylation status. Their activities may contribute to elevation of the PGC-1α protein through enhancing protein stability (26-28). It was not clear if this mechanism was activated by butyrate. To test this possibility, we examined activity of AMPK and p38 in the skeletal muscle. An increase in their phosphorylation was observed in the muscle lysate of butyrate-treated mice (FIG. 5A), suggesting increased activation of the two kinases by butyrate.

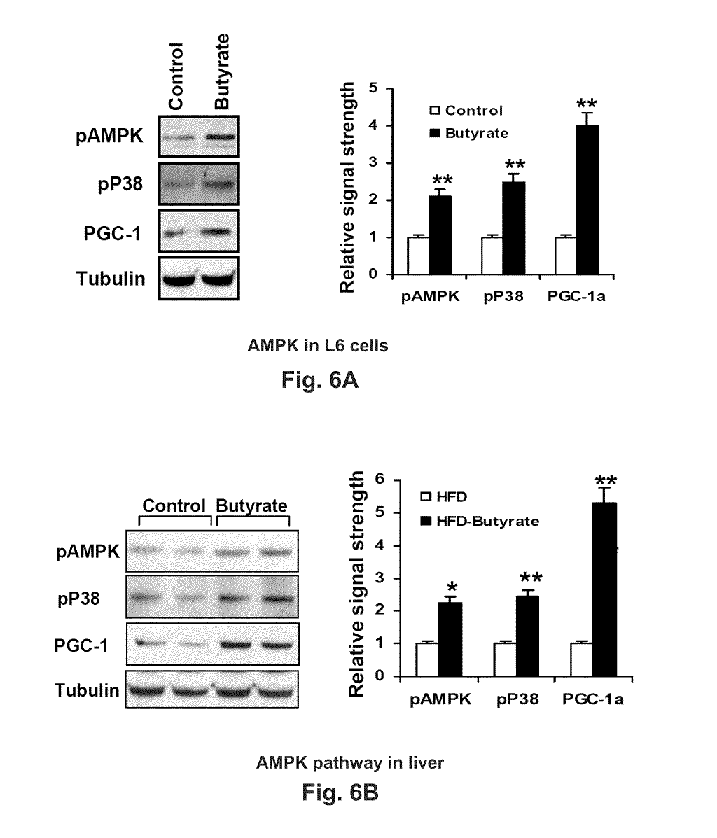

To further test the effect of butyrate on muscle, AMPK and PGC-1α were assayed in L6 muscle cells treated with butyrate. Differentiated L6 myotubes were starved in 0.25% BSA DMEM for overnight. The cells were treated with 500 μM of sodium butyrate for 4 hours and analyzed in an immunoblot. A representative immunoblot is shown in FIG. 6A (top). A mean value of triplicate experiments is shown in FIG. 6A (bottom). In the L6 cell line both AMPK and p38 phosphorylation were increased by butyrate in the cell culture (FIG. 6A), suggesting that butyrate is able to activate AMPK directly. In the same culture, the PGC-1α protein was increased (FIG. 6A).

AMPK and PGC-1α was also assayed in liver tissues. Whole cell lysates were prepared from liver tissues collected from mice on HFD for 13 weeks and analyzed in an immunoblot. In the experiments, pAMPK, p-p38 and PGC-1α were blotted with the specific antibodies. A representative blot is shown in FIG. 6B (top). A mean value of five mice is shown in the bar figure of FIG. 6B (bottom) (N=5). * P<0.05, ** P<0.001. In the liver of butyrate-treated mice, a similar pattern of changes was observed in AMPK, p38 and PGC-1α (FIG. 6B). These data consistently suggest that AMPK and p38 were activated by butyrate, and their activation may contribute to the increase in the PGC-1α activity.

### EXAMPLE 7

Effect of Dietary Butyrate on Mitochondrial Function.

Mitochondrial function was examined in the skeletal muscle tissue and L6 muscle cells under butyrate treatment. Fatty acid oxidation was monitored in the gastrocnemius muscle with ¹⁴C-labeled palmitic acid.

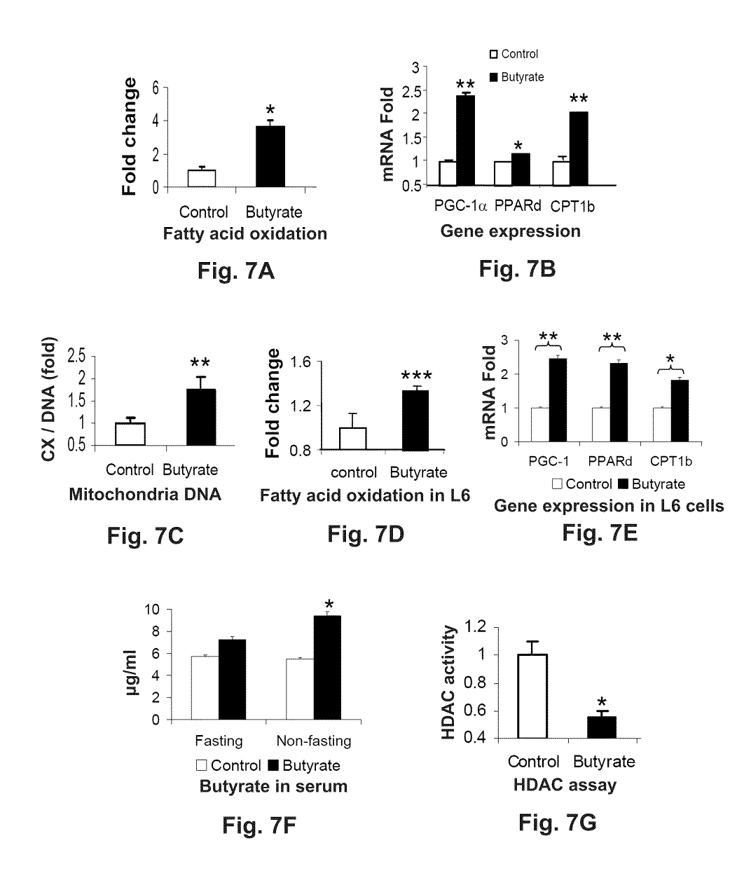

Vastus laterais muscle and blood samples were collected from mice at 13 weeks on HFD (18 weeks in age) and examined for fatty acid oxidation, gene expression, and blood lipids. FIG. 7A illustrates the fatty acid oxidation in muscle, measured as a fold change in ¹⁴C-labled CO₂. A 200% increase in ¹⁴C-labeled CO₂ was observed in the butyrate-treated mice, indicating an increase in mitochondrial function.

In addition, relative fold change in mRNA was used to indicate gene expression of PGC-1α target genes, such as CPT-1b (carnitine palmitoyltransferase-1b) and COX-I (cytochrome c oxidase I), and of the nuclear receptor PPARδ. The results are shown in FIGS. 7B and 7C. In FIG. 7C, mitochondrial DNA of COX I was determined by Sybr green RT-PCR. The fatty acid oxidation was associated with expression of PGC-1α target genes, such as CPT-1b (carnitine palmitoyltransferase-1b) and COX-I (cytochrome c oxidase I) (9). Expression of these two genes was increased in the skeletal muscle of butyrate-treated mice (FIGS. 7B and 7C). The nuclear receptor PPARδ promotes fatty acids oxidation in skeletal muscle (29). PPARδ expression was also increased in the butyrate-treated mice (FIG. 7B).

In addition, fully differentiated L6 cells were treated with 500 μM butyrate for 16 hours, and fatty acid oxidation was measured as a fold change in ¹⁴C-labled CO₂. The results are shown in FIG. 7D. FIG. 7E shows the relative change in mRNA expression for the three genes in FIG. 7B. Thus, in cultured L6 cells, a similar increase was observed in fatty acid oxidation and gene expression after butyrate treatment (FIGS. 7D and 7E). These data support the effect of butyrate in promotion of mitochondrial function.

### EXAMPLE 8

Butyrate Effect on Serum and HDAC Activity in Muscle.

The butyrate concentration was analyzed in plasma collected from the butyrate and control groups in mice fed HFD for 16 weeks. In a fasted condition (overnight fast), the butyrate concentration was 7.23±0.93 μg/ml in the butyrate group and 5.71±0.38 μg/ml in the control. In the fed condition, the butyrate concentration was 9.40±1.36 μg/ml in the butyrate group verses 5.48±0.60 μg/ml in the control (P<0.05, n=5) (FIG. 7F). The data indicate that dietary supplementation increased butyrate levels in the blood. Data are presented as means±SE (n=6). * P<0.05; ** P<0.001.

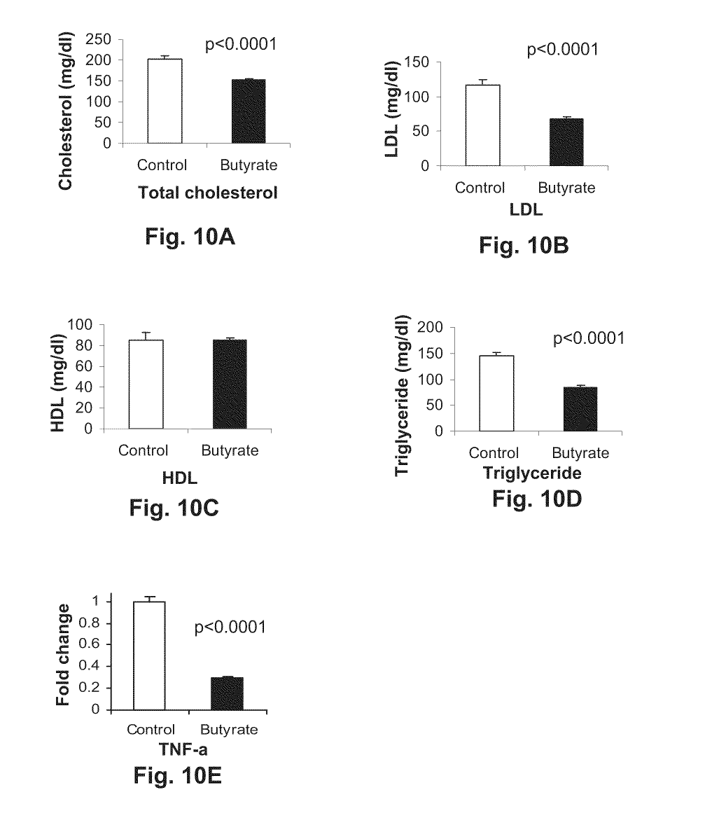

Without wishing to be bound by this theory, one activity of butyrate could be related to inhibition of HDAC. Sodium butyrate inhibits the class I and class II histone deacetylases (HDACs). To test this possibility, HDAC activity was examined in the skeletal muscle of mice at 16 weeks on HFD (FIG. 7G). The assay was conducted with the nuclear extracts of muscle samples. The HDAC activity was reduced by 50% in the butyrate group (FIG. 7G). TSA, a typical histone deacetylase inhibitor, was used as a positive control in the parallel treatment. The HDAC activity was decreased in the skeletal muscle of TSA-treated mice (see FIG. 10G). These data suggested that the dietary supplementation of butyrate leads to suppression of HDAC activity in the body.

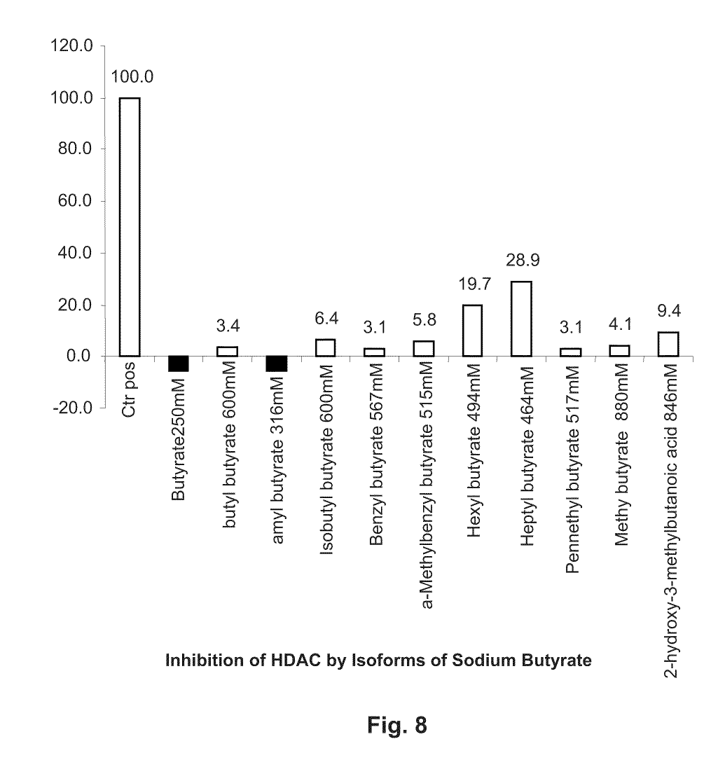

Several isoforms of butyrate were purchased (Sigma Aldrich Chemicals, St. Louis, Missouri) and tested for inhibitory activity of HDAC, using a Histone Deacetylase Assay Kit (Upstate Biotechnology, Lake Placid, N.Y.). All butyrate isoforms that were tested inhibited HDAC, which included butyrate, butyl butyrate, amyl butyrate, isobutyl butyrate, benzyl butyrate, a-methylbenzyl butyrate, hexyl butyrate, heptyl butyrate, pennetyl butyrate, methyl butyrate, and 2-hydroxy-3-methylbutanoic acid. The results are shown in FIG. 8. These isoforms have a more pleasant fruit smell than the smell of sodium butyrate. It is believed that all these isoforms of butyrate will be effective in increasing the insulin sensitivity and increasing the overall energy expenditure of mammals.

### EXAMPLE 9

Treatment of Obesity with Butyrate.

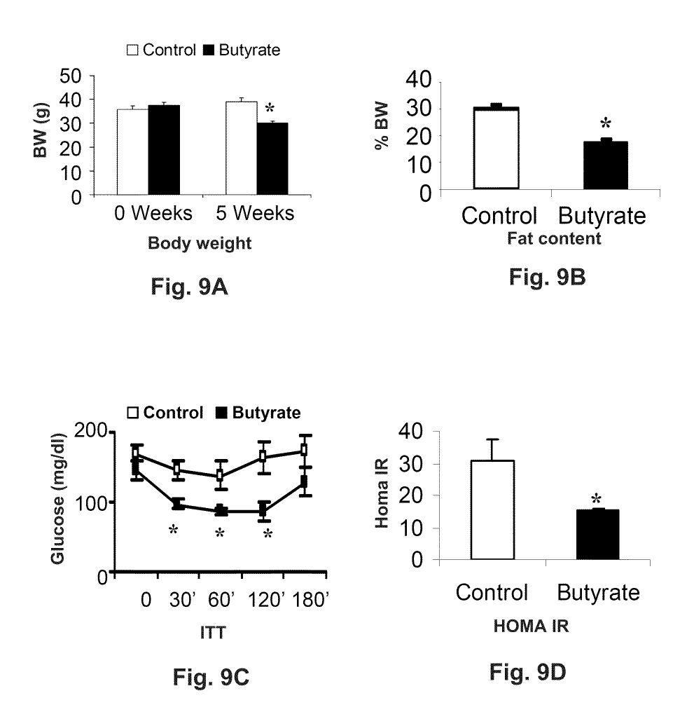

In the above examples, butyrate was administrated together with HFD during the induction of obesity. To test butyrate in the treatment of obesity and insulin resistance, butyrate was administered to obese mice that had been on HFD for 16 weeks. Obesity was induced in C57BL/6J mice fed on HFD for 16 weeks (21 weeks in age). The obese mice were then treated with butyrate through food supplement for 5 weeks. FIG. 9A shows body weight at the beginning and end of the 5 week butyrate treatment. After a 5-week treatment with butyrate, the obese mice lost 10.2% of their original body weight which dropped from 37.6 g to 34.4 g (FIG. 9A). In the control group, the body weight was increased by 15.8% (from 35.9 g to 41.6 g) during the same time period. Values are the means±SE (N=8 in each group). * P<0.05.

Fat content was determined in the body using NMR at the end of 5 week treatment with butyrate. Consistent with the change in body weight, the fat content was reduced by 10% in the butyrate group (FIG. 9B). At the end of 5 weeks, ITT was performed after 4 hours fast and HOMA IR was measured as described above. Further, the fasting glucose was reduced by 30% from 131 to 98.6 mg/dl (p<0.016), HOMA-IR was reduced by 50%, and ITT was improved significantly in the butyrate group (FIGS. 9C and 9D). Values are the means±SE (N=8 in each group). * P<0.05. These data suggested that butyrate is effective in the treatment of obesity and insulin resistance in the dietary obese model.

### EXAMPLE 10

Effect of Butyrate on Serum Lipids

Levels of cholesterol, total triglyceride and inflammation cytokines (TNF-a) were elevated in the blood of dietary obese mice. With 5% SB supplementation, these risk factors for cardiovascular disease were all reduced in the mice (FIG. 10A-E). Blood samples were taken from control and butyrate-supplemented mice at 10 weeks on HFD. The cholesterol, HDL (high density lipoprotein), LDL (low density lipoprotein), and triglyceride were measured with cholesterol reagent kit (cat #. 80015, Rainchem, San Diego, Calif.), and the Cardiochek whole blood test system (Polymer Technology Systems, Inc. Ind.). The results are shown in FIGS. 10A-10D. TNF-α was measured with Luminex (MADPK-71-03, Linco Research, Inc., St. Charles, Mo.) and the results shown in FIG. 10E. Data are presented as means±SEM (n=6). **P<0.001 by Student's t test. The results show that butyrate reduced total cholesterol by 25% (FIG. 10A), LDL by 41% (FIG. 10B), Triglyceride by 40% (FIG. 10D), and TNF-a by 70% (FIG. 10E), as compared with the control. These results suggest that dietary butyrate would have a beneficial effect in the prevention of cardiovascular disease.

### EXAMPLE 11

TSA, a HDAC Inhibitor, Effect on Energy Metabolism in Mice

Trichostatin A (TSA) is a well-established HDAC inhibitor that is frequently used in the study of HDACs. The data presented above indicate that the metabolic activity of butyrate may be in part related to inhibition of HDACs. TSA activity was examined in the same model of murine obesity. TSA was administrated through daily i.p. injection during HFD feeding to C57BL/6J mice. The control mice were injected with an identical volume of PBS.

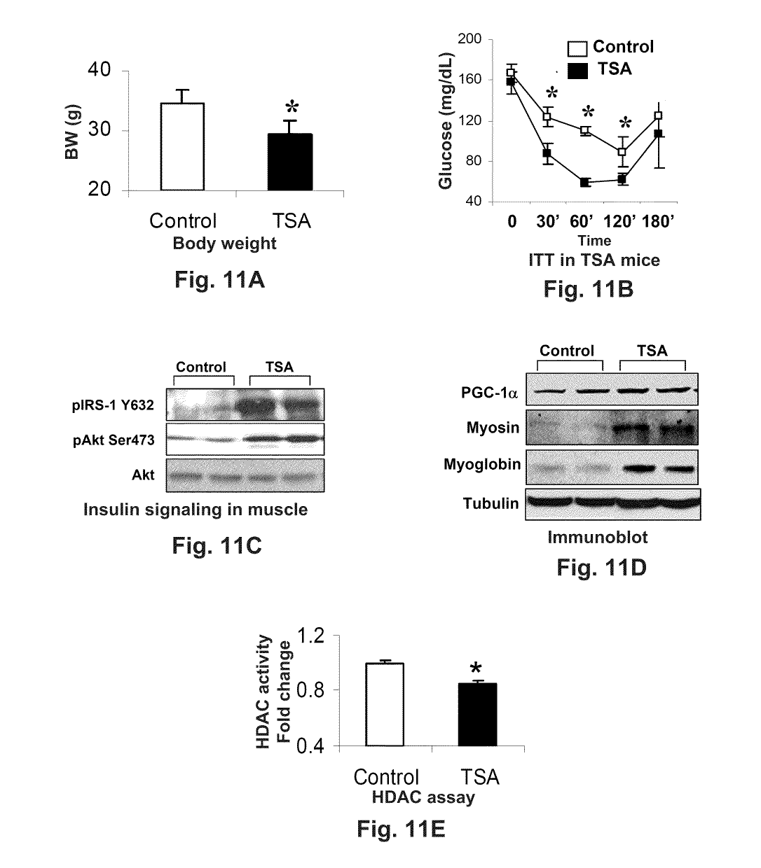

The mice at 5 weeks in age were fed HFD to induce obesity, and TSA was administrated at the dose of 0.6 ug/kg/day. The effects of TSA treatment were tested after 12-13 weeks. FIG. 11A shows the body weight at 12 weeks on HFD. The treated mice exhibited 16% less in body weight compared to the control at 12 weeks (FIG. 11A). FIG. 11B shows the results of ITT at 12 weeks on HFD (at 17 weeks of age), performed after 4 hour fast. FIG. 11C indicates the insulin signaling in the gastrocnemius muscle. The muscle was isolated after insulin (0.75 U/kg) injection for 30 minutes and used to prepare whole cell lysate for immunoblot. IRS-1 and Akt were examined for tyrosine (Y632) and serine (S473) phosphorylation. Insulin tolerance and insulin-induced signaling activities were significantly improved in the TSA-treated mice (FIGS. 11B and 11C).

In addition, the vastus laterais muscle was isolated from the mice at 13 weeks on HFD, and used to make serial cryostat sections of muscle. The muscle slides made from vastus lateralis, gastrocnemius (gastr.) and soleus muscle were stained with antibody against the type I myosin heavy chain in the oxidative fiber. The photograph was taken at 20× magnification. The whole cell lysate was prepared from gastrocnemius muscle and analyzed in an immunoblot. PGC-1α, type I myosin heavy chain (Myosin) and myoglobin were blotted with specific antibodies, and the results shown in FIG. 11D. The results in FIGS. 11A-11D are the mean±SE (n=8 in each group). *P<0.05 (vs. control).

The oxidative fiber ratio was increased in the skeletal muscle of TSA-treated mice. This was indicated by muscle color, histology and immunoblot results. The color of vastus laterais in TSA mice was deeper red than the control. (data not shown) Expression of the type I myosin heavy chain was increased in the gastrocnemius and soleus muscles, as shown by histology (data not shown). In the gastrocnemius muscle, protein levels for PGC-1α, the type I myosin heavy chain, and myoglobin were all increased in the immunoblot (FIG. 10D). The increase in PGC-1α may explain the elevated mitochondrial function in the skeletal muscle tissue of TSA-treated mice. These data suggest that TSA has a similar activity to butyrate in the regulation of fatty acid and glucose metabolism.

### EXAMPLE 12

Effect of 2.5% Butyrate on Mice on High Fat Diet

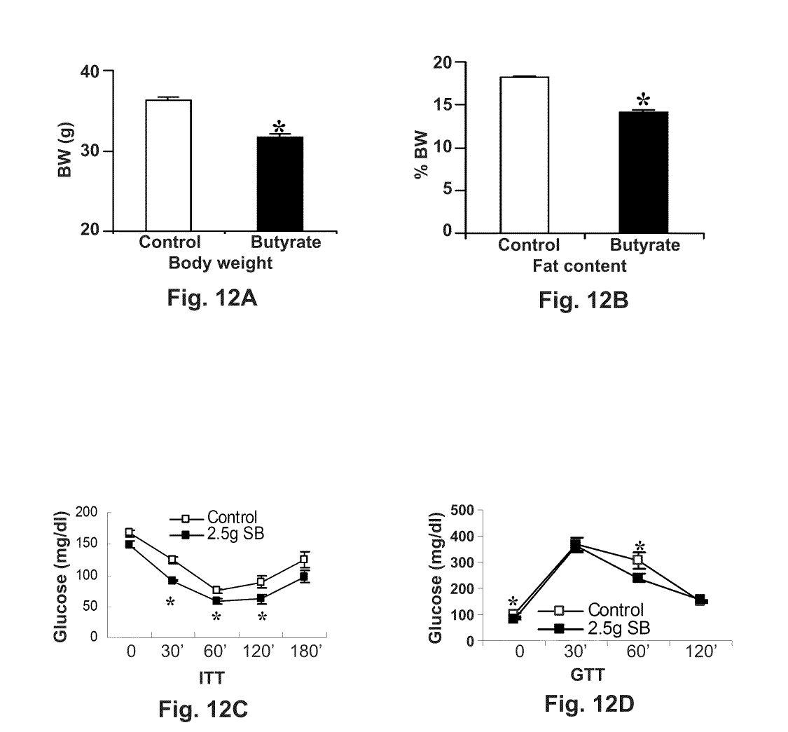

Sodium butyrate (#303410, Sigma) was tested at two dosages, 5% and 2.5% w/w in HFD. The metabolic effects presented above were from 5%. In the same mouse model, sodium butyrate exhibited similar activities at the low dosage 2.5% (FIGS. 12A-12D). After 12 week supplementation at 2.5%, butyrate reduced body weight and fat content in the 16 week-old mice significantly (FIGS. 12A and 12B). Insulin sensitivity at 12 weeks as indicated by insulin tolerance test (ITT) and glucose tolerance test (GTT) at 13 weeks was higher in the butyrate group than that of the control group (FIGS. 12C and 12D). For the glucose tolerance test, after an overnight fast, 2.5 g/kg glucose was injected intraperitoneally. Blood glucose was measured as described above at 0, 30, 60 and 120 min after injection. FIGS. 10A-10D show values as the means±SE (N=8 in each group), and * indicates p<0.05. These data indicate that sodium butyrate dietary supplementation is effective at the low dosage 2.5% w/w food.

### EXAMPLE 13

Dietary Butyrate Improved Insulin Sensitivity in Muscle, Fat and Liver.

The effect of dietary butyrate on whole body insulin sensitivity was assessed by looking at insulin effects on muscle, fat and liver tissues of control and butyrate mice. The insulin tolerance data indicated that butyrate protected insulin sensitivity in the diet-induced obesity mouse model. To understand which tissue contributes to the insulin sensitivity, a hyperinsulinemic and euglycemic clamp test was conducted in the diet-induced obese mouse.

Hyperinsulinemic-euglycemic clamps were performed at the Penn State Mouse Metabolic Phenotyping Center. The clamps were conducted in C57BL/6J mice at 12 weeks of age after 4 weeks on HFD with or without a 5% butyrate supplement. Following overnight fast (˜15 hour), a 2-hour hyperinsulinemic-euglycemic clamp was conducted in awake mice with a primed (150 mU/kg body weight) and continuous infusion of human regular insulin (Humulin; Eli Lilly, Indianapolis, Ind.) at a rate of 2.5 mU/kg/min to raise plasma insulin within a physiological range (38). Blood samples (20 μl) were collected at 20 min intervals for the immediate measurement of plasma glucose concentration, and 20% glucose was infused at variable rates to maintain glucose at basal concentrations. Basal and insulin-stimulated whole body glucose turnover were estimated with a continuous infusion of [3-³H]glucose (PerkinElmer, Boston, Mass.) for 2 hours prior to the clamps (0.05 μCi/min) and throughout the clamps (0.1 μCi/min), respectively. To estimate insulin-stimulated glucose uptake in individual tissues, 2-deoxy-D-[1-¹⁴C]glucose (2-[¹⁴C]DG) was administered as a bolus (10 μCi) at 75 min after the start of clamps. Blood samples were taken before, during, and at the end of clamps for the measurement of plasma [³H]glucose, ³H₂O, 2-[¹⁴C]DG concentrations, and/or insulin concentrations. At the end of the clamps, mice were euthanized, and tissues were taken for biochemical and molecular analysis.

Glucose concentrations during clamps were analyzed using 10 μl plasma by a glucose oxidase method on a Beckman Glucose Analyzer 2 (Beckman, Fullerton, Calif.). Plasma insulin concentrations were measured by ELISA using kits from Alpco Diagnostics (Salem, N.H.). Plasma concentrations of [3-³H]glucose, 2-[¹⁴C]DG, and ³H₂O were determined following deproteinization of plasma samples. The radioactivity of ³H in tissue glycogen was determined by digesting tissue samples in KOH and precipitating glycogen with ethanol. For the determination of tissue 2-[¹⁴]DG-6-P content, tissue samples were homogenized, and the supernatants were subjected to an ion-exchange column to separate 2-[¹⁴C]DG-6-P from 2-[¹⁴C]DG. Rates of basal hepatic glucose production (HGP) and insulin-stimulated whole body glucose turnover were determined as the ratio of the [³H]glucose infusion rate to the specific activity of plasma glucose at the end of the basal period and during the final 30 min of clamp, respectively (38). Insulin-stimulated rate of HGP during clamp was determined by subtracting the glucose infusion rate from whole body glucose turnover. Insulin-stimulated glucose uptake in individual tissues was assessed by determining the tissue content of 2-[¹⁴C]DG-6-P and plasma 2-[¹⁴C]DG profile.

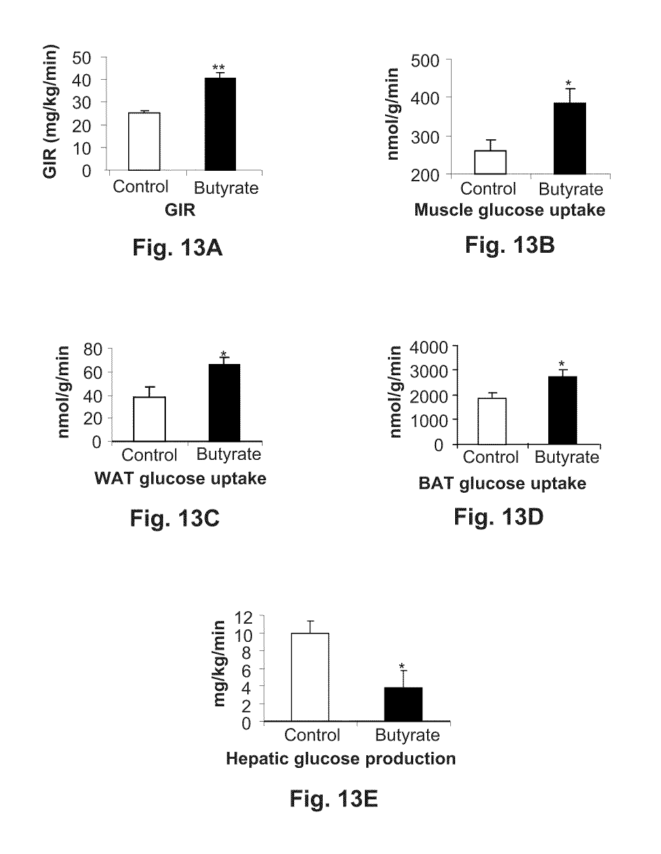

The averaged GIR (glucose infusion rate) during the last 40 min of clamps is presented in FIG. 13A. FIG. 13B shows the insulin-stimulated glucose uptake in skeletal muscle (gastrocnemius) during the clamps. FIG. 13C shows the insulin-stimulated glucose uptake in white adipose tissue (WAT), and FIG. 13D shows the insulin-stimulated glucose uptake in brown fat. FIG. 13E shows the hepatic glucose production (HGP) during clamps. Radiolabeled tracer was used to determine glucose uptake in peripheral tissues. In FIGS. 13A-13E, data are presented as means±SE (N=9). *P<0.05, **P<0.001 by Student's t test.

The data confirmed that insulin sensitivity was improved in the butyrate-treated mice by showing a 40% increase in glucose infusion rate (FIG. 13A). Glucose uptake was improved in muscle, white adipose tissue, and brown adipose tissue (FIGS. 13B-13D). Insulin sensitivity was also improved in the liver (FIG. 13E). These data indicate that butyrate improved the insulin sensitivity in all of the major tissues.

### EXAMPLE 14

Amyl Butyrate Reduced Adiposity and Improved Insulin Sensitivity in Mice:

Sodium butyrate has an unpleasant smell which may reduce its acceptability as a dietary supplement. As shown above, ten isoforms of butyrate were tested, and all of them inhibited HDAC similar to butyrate. All of the ten isoforms have more pleasant smell. Amyl Butyrate was tested in mice to see if it has similar metabolic effects in vivo as sodium butyrate. Amyl butyrate (W205915, Sigma-Aldrich) is a colorless clear liquid, and has an odor that is sweet and fruity, similar to that of banana, pineapple or cherry.

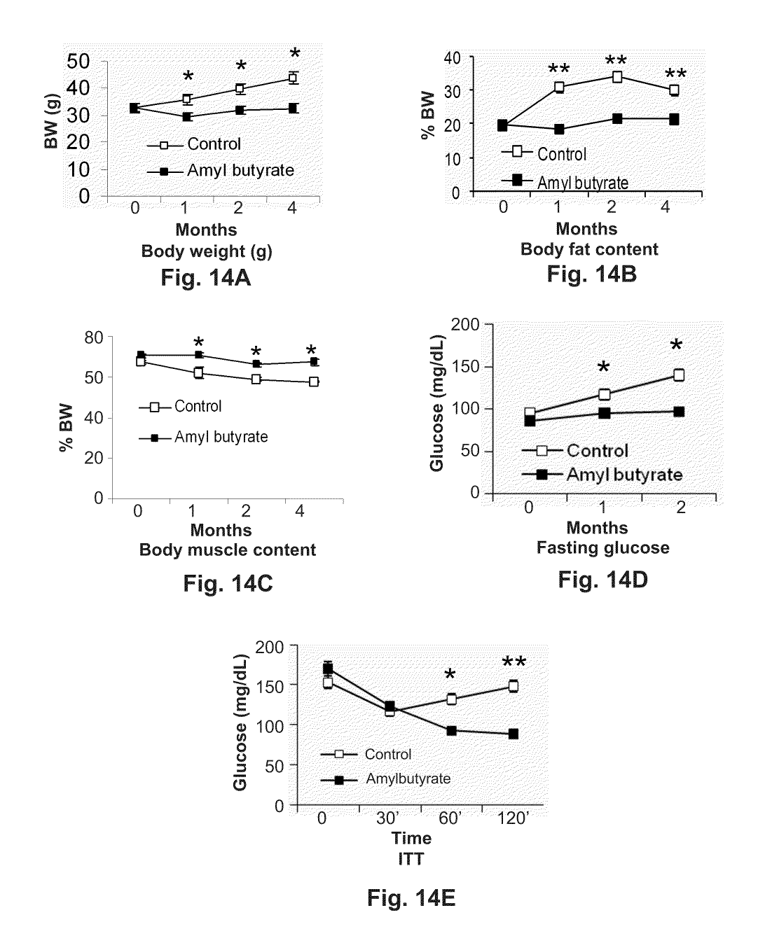

Amyl butyrate was administrated at 5 g/kg BW/day in C57BL/6J mice for 4 months. The amyl butyrate was added to the chow diet. FIG. 14A shows the body weight change over the four month period. When tested at the same dosage to sodium butyrate (5 g/Kg/day), Amyl butyrate prevented body weight gain in mice on chow diet (FIG. 14A). FIGS. 14B and 14C show the body fat and muscle content measured by NMR as described above. Amyl butyrate prevented fat gain without affecting lean body mass (FIGS. 14B and 14C). FIG. 14D shows the fasting glucose after an overnight fasting, and FIG. 14E shows the insulin tolerant test (ITT) after 4 months with the amyl butyrate treatment, and after 4 hours fasting. The amyl butyrate protected the mice from insulin resistance similar to the protection seen with sodium butyrate (FIGS. 14D and 14E). In FIGS. 14A-14E, values are the mean±SE (n=7). *P<0.05, **P<0.001 compared to the control by Student's t test. These data show that the isoforms of butyrate, e.g., amyl butyrate, show a similar effect on regulation of energy metabolism as seen with sodium butyrate.

After 4 month supplementation, the mice are protected from insulin resistance (FIGS. 14C and 14D). The data suggest that amyl butyrate has a similar activity to sodium butyrate in the regulation of metabolism.

### EXAMPLE 15

Effect of Tributyrin on Ob/Ob Mice

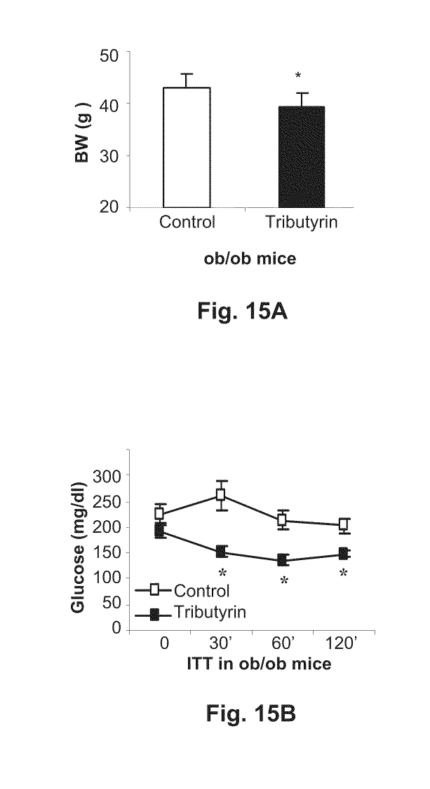

To test effect of different forms of butyric acid, tributyrin was used in the ob/ob genetically obese mice model. Tributyrin is formed by three molecules of butyric acid linked to one molecule of glycerol. Tributyrin was obtained from a commercial source (Sigma, Cat. #113026, St. Louis, Mo.). Tributyrin was used to supplement a chow diet at a dosage of 5 g/kg body weight/day. After 2 weeks, body weight was measured and an insulin tolerance test was given. The results are shown in FIGS. 15A and 15B. On the tributyrin diet, the weight gain was significantly reduced as compared with the control (FIG. 15A). Insulin sensitivity was preserved as indicated by the ITT results (FIG. 15B). These results indicate that tributyrin and butyrate have similar activity in the control of obesity and insulin resistance. In addition, the effect of butyrate was shown in two models of obesity, the dietary obesity mice on the HFD and the genetic obese mice (ob/ob).

Metabolic activities of butyric acid were examined in diet-induced obese mice. The most important observation is that butyrate supplementation at 5% w/w in HFD prevented development of dietary obesity and insulin resistance. It also reduced obesity and insulin resistance in obese mice. In the butyrate-treated mice, the plasma butyrate concentration was increased, and the blood lipids (triglycerides, cholesterol and total fatty acids) were decreased. The increase in energy expenditure and fatty acid oxidation may be responsible for the anti-obesity effect of butyrate. The butyrate supplementation did not reduce food intake, fat absorption or locomotor activity, suggesting that there was no toxicity from butyrate. Butyrate was tested at 5% and 2.5% w/w in the HFD in this study. At the lower (2.5% w/w) dosage, a similar metabolic activity was observed. At 5% in HFD, butyrate increased the calorie content from 58% to 64.4% in the fat. At the cellular level, butyrate increased mitochondrial respiration as indicated by the increase in oxygen consumption and carbon dioxide production. At the molecular level, an increased expression of PGC-1α, PPARδ and CPT1b may be involved in the stimulation of mitochondrial function by butyrate.

In vivo, butyrate was shown to be an activator of PGC-1α. The PGC-1α activity may be regulated by butyrate at three levels. The PGC-1α expression was increased in both mRNA and protein. The protein elevation was observed in brown fat, skeletal muscle and liver in the butyrate-treated mice. It may be a result of increased mRNA expression or extended half-life of the PGC-1α protein. The change in protein stability is supported by the activities of AMPK and p38 in tissues and cells after butyrate treatment. These kinases phosphorylate the PGC-1α protein and inhibit its degradation (27; 28; 31-34). As a transcriptional coactivator, the PGC-1α transcription activity may be induced by the phosphorylation, which leads to removal of a suppressor protein (p160 myb) that is associated with PGC-1α in the basal condition (35). p38 acts at the downstream of AMPK in the phosphorylation of PGC-1α (36). Therefore, AMPK may increase PGC-1α phosphorylation through direct and indirect (p38) mechanisms. Butyrate may act through induction of AMP levels in cells from an increased consumption of ATP (37). Induction of PGC-1α activity may be a molecular mechanism by which butyrate stimulates the mitochondrial function.

Inhibition of HDAC may contribute to the increased mRNA expression of PGC-1α, PPARδ and CPT1b. HDAC inhibition promotes gene expression through transcriptional activation, which is determined by the gene promoter activity. The promoter activation requires histone acetylation that opens the chromatin DNA to the general transcription factors for the transcription initiation and mRNA elongation. HDAC inhibits the gene promoter activity through deacetylation of the histone proteins. In the presence of butyrate, the promoter inhibition is prevented by the butyrate suppression of HDAC. The HDAC suppression will enhance the histone acetylation. This chromatin modification may occur in the gene promoters for PGC-1α, PPARδ and CPT1b for the up-regulation of gene transcription.

Butyrate induces type 1 fiber differentiation in the skeletal muscle. In skeletal muscle cells, inhibition of HDAC enhances myotube differentiation in vitro (28-30), and protects muscle from dystrophy in vivo (29-31). TSA, a typical histone deacetylase inhibitor, was tested in the parallel treatment with butyrate. TSA exhibited similar activity to butyrate in mice. TSA prevented dietary obesity, insulin resistance, and increased the type 1 fiber in the skeletal muscle. The activity was associated with elevation of PGC-1α protein.

In summary, dietary supplementation of butyrate can prevent and treat diet-induced obesity and insulin resistance in the mouse models of obesity. The mechanism of butyrate action is related to promotion of energy expenditure and induction of mitochondrial function. Stimulation of PGC-1α activity may be a molecular mechanism of the butyrate activity. Activation of AMPK and inhibition of HDACs may contribute to the PGC-1α regulation. Butyrate and its derivatives may have potential application in the prevention and treatment of metabolic syndrome in human.

### EXAMPLE 16

Dietary Butyrate Effect on Human Subjects

A clinical trial using human subjects will be conducted to test the effect of chronic addition of butyrate to the diet on metabolic rate and body fat. The dietary form of butyrate may be selected from the following: sodium butyrate or another butyrate salt, one of the butyrate isoforms known to inhibit HDAC, tributyrin, or a triglyceride with at least one butyrate attached to the glycerol, but more preferably two butyrates attached to the glycerol. The triglyceride can also have at least one long chain fatty acid (i.e., C16 or longer), for example oleate. The long chain fatty acids could include either an unsaturated or saturated fatty acid or a mixture. The clinical trial will be designed to show that incorporating butyrate-containing food fat into the diet of humans in doses proportional to those used above in mice will increase metabolic rate and reduce body fat.

Participants will be 8 healthy men or women between the ages of 18 and 70 years, inclusive with a body mass index (BMI) of 25 or greater. Subjects taking medications that could affect metabolic rate, subjects over 300 pounds, pregnant subjects, or subjects unwilling to avoid pregnancy during the study will be excluded. The butyrate will be incorporated into a food product with about 35 grams per dose, and the food will be checked for acceptability in taste and smell. The subjects will be randomized in a 1:1 ratio to receive the butyrate-rich food to be eaten three times a day or the equicaloric food made without butyrate. Subjects will have a DEXA scan during screening followed by a resting metabolic rate (RMR) using a flow-through metabolic hood. The subjects will then eat a dose of the food to which they were randomized and an RMR with RQ will be measured for the next 3 hours. The DEXA and RMR with RQ testing will be repeated during the last week of this 12 week study. The body fat, lean, percent body weight and BMI lost will be compared between the two groups by t-test, as will the area under the curve for metabolic rate and respiratory quotient. No adverse events or discomforts are anticipated. It is believed that a diet that is chronically supplemented with butyrate will cause an increase in metabolic rate, an increase in insulin sensitivity, and a decrease in body fat. In patients with a high BMI, it is believed that a diet chronically supplement with butyrate will cause weight loss and will preserve insulin sensitivity.

The term “therapeutically effective amount” as used herein refers to a daily dose of an amount of butyric acid (or its derivatives or isoforms) sufficient to increase either insulin sensitivity or metabolic rate of a mammal or to decrease body fat when taken for an extended period of time. The increase in insulin sensitivity or increase in metabolic rate or decrease in body fat should a statistically significant change (p<0.05). Methods to monitor insulin sensitivity, metabolic rate and body fat are well known to those skilled in the field and examples are taught in this specification. The dosage ranges for the administration of butyric acid are those that produce the desired effect, preferably from about 2% to about 10% wt/wt food, and more preferably from about 2.5% to about 5% wt/wt food. Generally, the dosage will vary with the age, weight, condition, and sex of the patient. A person of ordinary skill in the art, given the teachings of the present specification, may readily determine suitable dosage ranges. The dosage can be adjusted by the individual physician in the event of any contraindications. Moreover, butyric acid or its derivatives can be administered in pharmaceutically acceptable carriers known in the art. The application can be oral or by injection, with the preferred administration being oral.

Pharmaceutically acceptable carrier preparations for administration include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, emulsions or suspensions, including saline and buffered media. Parenteral vehicles include sodium chloride solution, Ringer's dextrose, dextrose and sodium chloride, lactated Ringer's, or fixed oils. The active therapeutic ingredient may be mixed with excipients that are pharmaceutically acceptable and are compatible with the active ingredient. Suitable excipients include water, saline, dextrose, and glycerol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

Butyric acid or its derivatives may be formulated into therapeutic compositions as pharmaceutically acceptable salts. These salts include the acid addition salts formed with inorganic acids such as, for example, hydrochloric or phosphoric acid, or organic acids such as acetic, oxalic, or tartaric acid, and the like. Salts also include those formed from inorganic bases such as, for example, sodium, potassium, ammonium, calcium or ferric hydroxides, and organic bases such as isopropylamine, trimethylamine, histidine, procaine and the like.

Butyric acid (or its derivatives) could be administered as tributyrin. Butyric acid is a small molecule that is absorbed when taken orally. Three butyric acid molecules (or its derivatives) could be attached to glycerol by ester bonds and would allow safe delivery of butyric acid without potential for an increase in acid or salt load. The dietary form of butyrate may also be a triglyceride with at least one butyrate attached to the glycerol, but more preferably two butyrates attached to the glycerol. The triglyceride can also have at least one long chain fatty acid (i.e., C16 or longer), for example oleate. The long chain fatty acids could include either an unsaturated or saturated fatty acid or a mixture. See, for example, U.S. Pat. No. 5,552,174 and U.S. Published Application No. 2004/0086621. Since esterases are abundant in the gastrointestinal tract and in tissue, the tributyrin or other triglycerides with butyrate should be rapidly broken down in the intestine.

### REFERENCES

1. Lagouge M, Argmann C, Gerhart-Hines Z, Meziane H, Lerin C, Daussin F, Messadeq N, Milne J, Lambert P, Elliott P, Geny B, Laakso M, Puigserver P, Auwerx J: Resveratrol Improves Mitochondrial Function and Protects against Metabolic Disease by Activating SIRT1 and PGC-1alpha. Cell, 2006
2. Baur J A, Pearson K J, Price N L, Jamieson H A, Lerin C, Kalra A, Prabhu V V, Allard J S, Lopez-Lluch G, Lewis K, Pistell P J, Poosala S, Becker K G, Boss O, Gwinn D, Wang M, Ramaswamy S, Fishbein K W, Spencer R G, Lakatta E G, Le Couteur D, Shaw R J, Navas P, Puigserver P, Ingram D K, de Cabo R, Sinclair D A: Resveratrol improves health and survival of mice on a high-calorie diet. Nature 444:337-342, 2006
3. Watanabe M, Houten S M, Mataki C, Christoffolete M A, Kim B W, Sato H, Messaddeq N, Harney J W, Ezaki O, Kodama T, Schoonjans K, Bianco A C, Auwerx J: Bile acids induce energy expenditure by promoting intracellular thyroid hormone activation. Nature 439:484-489, 2006
4. Zhang C Y, Parton L E, Ye C P, Krauss S, Shen R, Lin C T, Porco J A, Jr., Lowell B B: Genipin inhibits UCP2-mediated proton leak and acutely reverses obesity- and high glucose-induced beta cell dysfunction in isolated pancreatic islets. Cell Metab 3:417-427, 2006
5. Pryde S E, Duncan S H, Hold G L, Stewart C S, Flint H J: The microbiology of butyrate formation in the human colon. FEMS Microbiol Lett 217:133-139, 2002
6. Roy C C, Kien C L, Bouthillier L, Levy E: Short-chain Fatty acids: ready for prime time? Nutr Clin Pract 21:351-366, 2006
7. Davie J R: Inhibition of Histone Deacetylase Activity by Butyrate. J. Nutr. 133:2485S-2493, 2003
8. Lin J, Handschin C, Spiegelman B M: Metabolic control through the PGC-1 fAmyly of transcription coactivators. Cell Metab 1:361-370, 2005
9. Lin J, Wu H, Tan P T, Zhang C Y, Wu Z, Boss O, Michael L F, Puigserver P, Isotani E, Olson E N, Lowell B B, Bassel-Duby R, Spiegelman B M: Transcriptional co-activator PGC-1 alpha drives the formation of slow-twitch muscle fibres. Nature 418:797-801, 2002
10. Puigserver P, Wu Z, Park C W, Graves R, Wright M, Spiegelman B M: A cold-inducible coactivator of nuclear receptors linked to adaptive thermogenesis. Cell 92:829-839, 1998
11. Mootha V K, Handschin C, Arlow D, Xie X, St Pierre J, Sihag S, Yang W, Altshuler D, Puigserver P, Patterson N, Willy P J, Schulman I G, Heyman R A, Lander E S, Spiegelman B M: Erralpha and Gabpa/b specify PGC-1alpha-dependent oxidative phosphorylation gene expression that is altered in diabetic muscle. Proc Natl Acad Sci USA 101:6570-6575, 2004
12. Mootha V K, Lindgren C M, Eriksson K F, Subramanian A, Sihag S, Lehar J, Puigserver P, Carlsson E, Ridderstrale M, Laurila E, Houstis N, Daly M J, Patterson N, Mesirov J P, Golub T R, Tamayo P, Spiegelman B, Lander E S, Hirschhorn J N, Altshuler D, Groop L C: PGC-1alpha-responsive genes involved in oxidative phosphorylation are coordinately downregulated in human diabetes. Nat Genet 34:267-273, 2003
13. Petersen K F, Befroy D, Dufour S, Dziura J, Ariyan C, Rothman D L, DiPietro L, Cline G W, Shulman G I: Mitochondrial dysfunction in the elderly: possible role in insulin resistance. Science 300:1140-1142, 2003
14. Patti M E, Butte A J, Crunkhorn S, Cusi K, Berria R, Kashyap S, Miyazaki Y, Kohane I, Costello M, Saccone R, Landaker E J, Goldfine A B, Mun E, DeFronzo R, Finlayson J, Kahn C R, Mandarino L J: Coordinated reduction of genes of oxidative metabolism in humans with insulin resistance and diabetes: Potential role of PGC1 and NRF1. PNAS 100:8466-8471, 2003
15. Gao Z, Wang Z, Zhang X, Butler A A, Zuberi A, Gawronska-Kozak B, Lefevre M, York D, Ravussin E, Berthoud H R, McGuinness O, Cefalu W T, Ye J: Inactivation of PKC {theta} leads to increased susceptibility to obesity and dietary insulin resistance in mice Am J Physiol Endocrinol Metab 292:E84-91, 2007
16. Ye J, Gao Z, Yin J, He H: Hypoxia is a potential risk factor for chronic inflammation and adiponectin reduction in adipose tissue of ob/ob and dietary obese mice. Am J Physiol Endocrinol Metab 293:E1118-E1128, 2007
17. Ye J, Cippitelli M, Dorman L, Ortaldo J R, Young H A: The nuclear factor YY1 suppresses the human gamma interferon promoter through two mechanisms: inhibition of AP1 binding and activation of a silencer element. Mol Cell Biol 16:4744-4753, 1996
18. O'Fallon J V, Busboom J R, Nelson M L, Gaskins C T: A direct method for fatty acid methyl ester synthesis: application to wet meat tissues, oils, and feedstuffs. J Anim Sci 85:1511-1521, 2007
19. Schwarz M, Lund E G, Setchell K D, Kayden H J, Zerwekh J E, Bjorkhem I, Herz J, Russell D W: Disruption of cholesterol 7alpha-hydroxylase gene in mice. II. Bile acid deficiency is overcome by induction of oxysterol 7alpha-hydroxylase. J Biol Chem 271:18024-18031, 1996
20. Rothwell N J, Stock M J: A role for brown adipose tissue in diet-induced thermogenesis. Nature 281:31-35, 1979
21. Brooks S L, Rothwell N J, Stock M J, Goodbody A E, Trayhurn P: Increased proton conductance pathway in brown adipose tissue mitochondria of rats exhibiting diet-induced thermogenesis. Nature 286:274-276, 1980
22. Glick Z, Teague R J, Bray G A: Brown adipose tissue: thermic response increased by a single low protein, high carbohydrate meal. Science 213:1125-1127, 1981
23. Jung R T, Shetty P S, James W P, Barrand M A, Callingham B A: Reduced thermogenesis in obesity. Nature 279:322-323, 1979
24. Puigserver P, Spiegelman B M: Peroxisome Proliferator-Activated Receptor-gamma Coactivator 1alpha (PGC-1alpha): Transcriptional Coactivator and Metabolic Regulator. Endocr Rev 24:78-90, 2003
25. Bassel-Duby R, Olson E N: Signaling pathways in skeletal muscle remodeling. Annu Rev Biochem 75:19-37, 2006
26. Jager S, Handschin C, St-Pierre J, Spiegelman B M: AMP-activated protein kinase (AMPK) action in skeletal muscle via direct phosphorylation of PGC-1alpha. Proc Natl Acad Sci USA 104:12017-12022, 2007
27. Knutti D, Kressler D, Kralli A: Regulation of the transcriptional coactivator PGC-1 via MAPK-sensitive interaction with a repressor. Proc Natl Acad Sci USA 98:9713-9718, 2001
28. Puigserver P, Rhee J, Lin J, Wu Z, Yoon J C, Zhang C Y, Krauss S, Mootha V K, Lowell B B, Spiegelman B M: Cytokine stimulation of energy expenditure through p38 MAP kinase activation of PPARgamma coactivator-1. Mol Cell 8:971-982, 2001
29. Wang Y X, Lee C H, Tiep S, Yu R T, Ham J, Kang H, Evans R M: Peroxisome-Proliferator-Activated Receptor delta Activates Fat Metabolism to Prevent Obesity. Cell 113:159-170, 2003
30. Sacks F M, Bray G A, Carey V J, Smith S R, Ryan D H, Anton S D, McManus K, Champagne C M, Bishop L M, Laranjo N, Leboff M S, Rood J C, de Jonge L, Greenway F L, Loria C M, Obarzanek E, Williamson D A: Comparison of weight-loss diets with different compositions of fat, protein, and carbohydrates. N Engl J Med 360:859-873, 2009
31. Irrcher I, Adhihetty P J, Sheehan T, Joseph A M, Hood D A: PPARgamma coactivator-1alpha expression during thyroid hormone- and contractile activity-induced mitochondrial adaptations. Am J Physiol Cell Physiol 284:C1669-1677, 2003
32. Suwa M, Nakano H, Kumagai S: Effects of chronic AICAR treatment on fiber composition, enzyme activity, UCP3, and PGC-1 in rat muscles. J Appl Physiol 95:960-968, 2003
33. Terada S, Goto M, Kato M, Kawanaka K, Shimokawa T, Tabata I: Effects of low-intensity prolonged exercise on PGC-1 mRNA expression in rat epitrochlearis muscle. Biochem Biophys Res Commun 296:350-354, 2002
34. Zong H, Ren J M, Young L H, Pypaert M, Mu J, Birnbaum M J, Shulman G I: AMP kinase is required for mitochondrial biogenesis in skeletal muscle in response to chronic energy deprivation. Proc Natl Acad Sci USA 99:15983-15987, 2002
35. Fan M, Rhee J, St-Pierre J, Handschin C, Puigserver P, Lin J, Jaeger S, Erdjument-Bromage H, Tempst P, Spiegelman B M: Suppression of mitochondrial respiration through recruitment of p160 myb binding protein to PGC-1alpha: modulation by p38 MAPK. Genes Dev 18:278-289, 2004
36. Xi X, Han J, Zhang J-Z: Stimulation of Glucose Transport by AMP-activated Protein Kinase via Activation of p38 Mitogen-activated Protein Kinase. J. Biol. Chem. 276:41029-41034, 2001
37. Beauvieux M C, Tissier P, Gin H, Canioni P, Gallis J L: Butyrate impairs energy metabolism in isolated perfused liver of fed rats. J Nutr 131:1986-1992, 2001
38. Kim, H J, Higashimori T, Park S Y, Choi H, Dong J, Kim Y J, Noh H L, Cho Y R., Cline G, Kim Y B, and Kim J K: Differential effects of interleukin-6 and -10 on skeletal muscle and liver insulin action in vivo. Diabetes 53:1060-1067, 2004.
39. Dradkley, J K, Zhang Y, Amaral D M, and Young J W: Metabolic effects of intraruminal administration of 1,3-butanediol or tributyrin in lactating goats. J Dairy Sci 72:1986-1995, 1989.
40. Mineo H, Kanal M, Kato S and Ushijima J I: Effects of intravenous injection of butyrate, valerate and their isomers on endocrine pancreatic responses in conscious sheet (Ovis Aries). Comp Biochem Physiol 95A:411-416, 1990.
41. Gürtler H, Gálfi P, Fürll M., Neogrády S, Kutas F and Schäfer M: Acute metabolic and hormonal effects of intravenously administered sodium n-butyrate in untreated and alloxan-diabetic sheep. J Vet Med A 40:371-383, 1993.
42. Hamada T, Hodate K, Matsumoto M and Ishii T: Counteractive effects of propionate or 1,2-propanediol against hypoglycemia and ketonemia of tributyrin-treated cows. J Dairy Sci 67:1452-1456, 1984.
43. Galfi P and Bokori J: Feeding trial in pigs with a diet containing sodium n-butyrate. Acta Vet Hung 38:3-17, 1990.
44. Liu H K, Green B D, Flatt P R, McClenaghan N H, and McCluskey J T: Effects on long-term exposure to nicotinamide and sodium butyrate on growth, viability, and the function of clonal insulin secreting cells. Endocrine Research 30:61-68, 2004.

The complete disclosures of all references cited in this specification are hereby incorporated by reference. Also made an integral part of this specification are the complete disclosures of the following documents: Z. Gao et al., “Butyrate Improves Insulin Sensitivity through PGC1α-Mediated Energy Expenditure in Mice,” a manuscript submitted to Cell Metabolism on Jan. 7, 2008; D. Y. Jung et al., “Chronic Butyrate Treatment Protects Mice from Developing High-Fat Diet-Induced Obesity and Insulin Resistance,” an abstract submitted for the American Diabetes Association 2008 Meeting, San Francisco, California, Jun. 8-10, 2008; and Z. Gao et al., “Butyrate Improves Insulin Sensitivity and Increases Energy Expenditure in Mice,” Diabetes, vol. 58(7), 1509-1527 (2009), epub Apr. 14, 2009.

What is claimed:

1. A method to increase the energy expenditure in a non-ruminant mammal, said method comprising chronically administering to the mammal a therapeutically effective dose of a compound selected from the group consisting of butyric acid and its isoforms.

2. A method to increase the sensitivity of a non-ruminant mammal to insulin, said method comprising chronically administering to the mammal a therapeutically effective dose of a compound selected from the group consisting of butyric acid and its isoforms.

3. A method to decrease the amount of body fat in a non-ruminant mammal, said method comprising chronically administering to the mammal a therapeutically effective dose of a compound selected from the group consisting of butyric acid and its isoforms.

4. A method to decrease the body weight of a non-ruminant mammal, said method comprising chronically administering to the mammal a therapeutically effective dose of a compound selected from the group consisting of butyric acid and its isoforms.

5. A method to increase PGC-1α activity of a non-ruminant mammal, said method comprising chronically administering to the mammal a therapeutically effective dose of of a compound selected from the group consisting of butyric acid and its isoforms.

6. A method to increase AMPK activity of a non-ruminant mammal, said method comprising chronically administering to the mammal a therapeutically effective dose of of a compound selected from the group consisting of butyric acid and its isoforms.

7. A method to increase type I fiber (oxidative fiber) in skeletal muscle of a non-ruminant mammal, said method comprising chronically administering to the mammal a therapeutically effective dose of a compound selected from the group consisting of butyric acid and its isoforms.

8. A method to increase fatty acid oxidation in a non-ruminant mammal, said method comprising chronically administering to the mammal a therapeutically effective dose of a compound selected from the group consisting of butyric acid and its isoforms.

9. A method to prevent diet-induced obesity in a non-ruminant mammal on a high fat diet, said method comprising chronically administering to the mammal a therapeutically effective dose of a compound selected from the group consisting of butyric acid and its isoforms.

10. A method to prevent diet-induced insulin resistance in a non-ruminant mammal on a high fat diet, said method comprising chronically administering to the mammal a therapeutically effective dose of a compound selected from the group consisting of butyric acid and its isoforms.

11. A method as in any of claims 1-10, in which the butyric acid isoforms are one or more isoforms selected from the group consisting of butyl butyrate, amyl butyrate, isobutyl butyrate, benzyl butyrate, a-methylbenzyl butyrate, hexyl butyrate, heptyl butyrate, pennetyl butyrate, methyl butyrate, and 2-hydroxy-3-methylbutanoic acid.

12. The method as in any of claims 1-10, wherein the compound is administered in the form of a triglyceride.

13. The method as in any of claims 1-10, wherein the dose of the compound administered orally is from about 2% to about 10% wt/wt total food intake of the mammal.

14. The method as in any of claims 1-10, wherein the dose of the compound administered orally is from about 2.5% to about 5% wt/wt total food intake of the mammal.
